(19) United States
(12) Patent Application Publication
(10) Pub. No.: US 2026/0273252 A1
(43) Pub. Date: Sep. 17, 2026

(54) GROOVED MICRONEEDLES FOR PASSIVE AND ACTIVE DRUG DELIVERY

(71) Applicant: Trustees of Tufts College, Medford, MA (US)

(72) Inventor: Sameer Sonkusale, Medford, MA (US)

(21) Appl. No.: 19/167,112

(22) PCT Filed: Mar. 21, 2024
(86) PCT No.: PCT/US2024/020871

§ 371 Date: Sep. 19, 2025

Related U.S. Application Data
(60) Provisional application No. 63/491,518, filed on Mar. 21, 2023.

(51) Int. Cl. A61M 37/00

(52) U.S. Cl. CPC A61M 37/0015; A61M 2037/0023; A61M 2037/0038; A61M 2037/0061

(57) ABSTRACT

Disclosed herein are systems and methods for delivering a fluid containing a drug solution to a region of interest of a subject. The system includes a patch including at least one microneedle coupled thereto, a reservoir coupled to the patch and retaining a fluid containing a drug solution therein, and a pump coupled to the reservoir. The at least one grooved microneedle defines a fluid channel therein, and the pump is configured to direct the fluid containing the drug solution from the reservoir, through the fluid channel of the microneedle, and to a region of interest of a subject.

### CROSS-REFERENCE TO RELATED APPLICATIONS

This application claims priority to and the benefit of U.S. Provisional Patent Application No. 63/491,518, filed on Mar. 21, 2023, which is incorporated herein by reference in its entirety.

### STATEMENT REGARDING FEDERALLY FUNDED RESEARCH

Not Applicable.

### BACKGROUND

The present application generally relates to drug delivery systems and methods and relates in particular to autonomous drug delivery systems and methods.

Many systemic drugs require administration by injection, e.g., intravenous, intramuscular, or subcutaneous injection. This is also true of certain therapeutic agents, e.g., vaccines, and diagnostic agents, e.g., radiographic contrast. Administration by injection is often required for drugs with poor or unpredictable absorption by mouth or other routes. This is typically the case with high-molecular-weight drugs, e.g., proteins including therapeutic monoclonal antibodies, because they are degraded by the oral route and not absorbed. Some low-molecular-weight drugs, such as propranolol, morphine, vancomycin, and tetrahydrocannabinol (THC) are also poorly or inconsistently absorbed by the oral route. In some cases, first-pass metabolism has been found to interfere with the oral absorption of many drugs, thus inconsistent absorption as discussed above.

Further, drug administration by injection is painful and often requires the services of a healthcare provider, which is expensive and inconvenient for the patient. Intranasal administration routes offer some advantages over administration by injection, but the low intrinsic permeability of hydrophilic medications, rapid mucociliary clearance, and enzyme degradation severely impact drug absorption associated with this method.

To solve the foregoing problems, there has been much interest in delivering drugs across the skin, i.e., transdermally. Transdermal administration remains relatively uncommon, however, due to a technical challenge: the epidermis—and particularly the most superficial layer thereof, i.e., the stratum corneum—is a formidable barrier to molecules weighing more than about 500 Daltons, and molecules with certain physicochemical properties, e.g., high hydrophilicity or hydrophobicity.

One technical approach to deliver a drug past the epidermis for transdermal absorption is to use drug-loaded, skin-penetrating microneedles. Generally, these microneedles comprise: (1) a rigid structure to pierce into the skin; and (2) a drug active ingredient, typically combined with one or more excipients. Microneedles offer a convenient transdermal delivery route with potential for long-term sustained release of drugs. In recent years, Microneedles have received attention as a way to avoid degradation of drugs in the gastrointestinal tract, first-pass effects of the liver associated with oral delivery, and the pain and inconvenience of intravenous injection. In addition, using microneedles has offered a minimally invasive, less painful, and self-administrable delivery of drugs ranging from cosmetics to vaccinations which has made microneedles even more attractive.

Traditional microneedles may be categorized as solid, hollow, dissolving, merged tip, and porous. However, it has been a challenge to make hard hollow microneedles that is fabricated reliably and in a cost-effective manner. Furthermore, most other current microneedle technologies (e.g., hydrogel microneedles) do not have mechanical properties for reliable and stable skin penetration. Moreover, most microneedles can only carry limited amounts of drug. This limitation is particularly problematic for drugs requiring excipients, especially solvents. It is also problematic for drugs that require high absolute doses, e.g., high-molecular-weight drugs, low-potency drugs, and antibiotics. Further, some types of traditional microneedles are physically or chemically incompatible with some drugs.

Therefore, there exists a need for systems and methods of using and manufacturing microneedles to create an efficient and economical drug delivery system. More specifically, there exists a need for improved microneedle drug delivery systems that overcome one or more of the obstacles associated with traditional drug delivery techniques, as discussed above.

### SUMMARY

The present disclosure overcomes the aforementioned drawbacks by providing systems and methods for creating efficient, compact microneedle arrays capable of delivering a suitable dose of a drug to a surface region of interest of a subject. The systems and methods provided herein are achieved in a cost and time efficient manner compared to traditional systems and methods.

In accordance with some aspects of the present disclosure, a drug delivery system is provided. The system includes a patch including at least one microneedle coupled thereto, a reservoir coupled to the patch and retaining a fluid containing a drug solution therein, and a pump coupled to the reservoir. The at least one grooved microneedle defines a fluid channel therein, and the pump is configured to direct the fluid containing the drug solution from the reservoir, through the fluid channel of the microneedle, and to a region of interest of a subject.

In accordance with some aspects of the present disclosure, a system for delivering a fluid containing a drug solution to a region of interest of a subject is provided. The system includes a patch including at least one microneedle coupled to a first side thereof, and a reservoir coupled to a second side of the patch. The at least one microneedle includes a body with a central channel therein, the central channel being in fluid communication with a groove disposed on an exterior side of the body.

In accordance with some aspects of the present disclosure, a method of transdermal drug delivery is provided. The method includes placing, on a subject's skin, a microneedle patch including at least one microneedle coupled to a first side of the microneedle patch and a reservoir coupled to a second side of the microneedle patch. The method further includes driving a fluid containing a drug solution from the reservoir, through a fluid channel defined by the at least one microneedle, and to a region of interest in a subject.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A is a schematic diagram of an example drug delivery system using a grooved microneedle array, in accordance with some aspects of the present disclosure.

FIG. 1B is a schematic diagram of another example drug delivery system including a micropump, in accordance with some aspects of the present disclosure.

FIG. 2A is a perspective view of a grooved microneedle array including an absorbent bead in an initial non-expanded state, in accordance with some aspects of the present disclosure.

FIG. 2B is a perspective view of the microneedle array of FIG. 2A with the absorbent bead in an intermediate state.

FIG. 2C is a perspective view of the microneedle array of FIG. 2A with the absorbent bead in a final expanded state.

FIG. 3 is a flowchart of non-limiting example steps for a method of delivering a drug using a microneedle patch, in accordance with some aspects of the present disclosure.

FIG. 4A is a top view of a symmetric cross-over-lines (COL) laser engraving pattern used to form solid microneedle tips, in accordance with some aspects of the present disclosure.

FIG. 4B is perspective view of the symmetric COL laser engraving pattern of FIG. 4A.

FIG. 4C is a top view of an asymmetric COL laser engraving pattern used to form grooved microneedle tips, in accordance with some aspects of the present disclosure.

FIG. 4D is a perspective view of the asymmetric COL laser engraving pattern of FIG. 4C.

FIG. 5A is a rear side view of an example grooved microneedle, in accordance with some aspects of the present disclosure.

FIG. 5B is a front side view of the example grooved microneedle of FIG. 5A.

FIG. 5C is a top plan view of the example grooved microneedle of FIG. 5A.

FIG. 5D is an isometric view of the example grooved microneedle of FIG. 5A.

FIG. 5E is a perspective view of a molded microneedle that is manufactured based on the design of the example grooved microneedle of FIG. 5A.

FIG. 5F is a perspective view of an example grooved microneedle array, in accordance with some aspects of the present disclosure.

FIG. 5G is a perspective view of a molded microneedle array that is manufactured based on the design of the example grooved microneedle array of FIG. 5F.

FIG. 6A is an isometric view of another example grooved microneedle including a central channel, in accordance with some aspects of the present disclosure.

FIG. 6B is a perspective view of an example molded microneedle that is manufactured based on the design of the example grooved microneedle of FIG. 6A.

FIG. 6C is a top view of another example molded microneedle array including microneedles similar to the molded microneedle of FIG. 6B.

FIG. 6D is a detail perspective view of the example molded microneedle array of FIG. 6C.

FIG. 7 is a preparation scheme of a side cross-sectional view of a grooved microneedle array, in accordance with some aspects of the present disclosure.

FIG. 8A is an image of yet another example grooved microneedle array, in accordance with some aspects of the present disclosure.

FIG. 8B is a detail image of the example grooved microneedle array of FIG. 8A.

FIG. 9A is an image of the fluid extraction behavior of a grooved microneedle array with expandable fluid retention beads, in accordance with some aspects of the present disclosure.

FIG. 9B is a graph illustrating height and width changes of the bead of FIG. 9A over time during fluid extraction.

FIG. 10A is a schematic diagram illustrating a COL laser engraving method to create a grooved microneedle mold, in accordance with some aspects of the present disclosure.

FIG. 10B is a schematic diagram illustrating a molding method for fabricating grooved microneedles, in accordance with some aspects of the present disclosure.

FIG. 11 is a schematic diagram illustrating methods of fabricating a drug delivery system including grooved microneedles and a storage reservoir, in accordance with some aspects of the present disclosure.

FIG. 12 is an image of an electronic chip in an adhesive bandage, in accordance with some aspects of the present disclosure.

FIG. 13A is a top view of an image of a grooved microneedle array with a channel width of 50 μm, in accordance with some aspects of the present disclosure.

FIG. 13B is a top view of an image of a grooved microneedle array with a channel width of 100 μm, in accordance with some aspects of the present disclosure.

FIG. 13C is a top view of an image of a grooved microneedle array with a channel width of 200 μm, in accordance with some aspects of the present disclosure.

FIG. 13D is a side view of the grooved microneedle array of FIG. 13A.

FIG. 13E is a side view of the grooved microneedle array of FIG. 13B.

FIG. 13F is a side view of the grooved microneedle array of FIG. 13C.

FIG. 14A is an image of an agar gel before sulforhodamine B dye is passively delivered via a grooved microneedle array with a channel width of 50 μm, in accordance with some aspects of the present disclosure.

FIG. 14B is an image of an agar gel before sulforhodamine B dye is passively delivered via a grooved microneedle array with a channel width of 100 μm, in accordance with some aspects the present disclosure.

FIG. 14C is an image of an agar gel before sulforhodamine B dye is passively delivered via a grooved microneedle array with a channel width of 200 μm, according to the present disclosure.

FIG. 14D is an image of the agar gel illustrated in FIG. 14A after sulforhodamine B dye has been passively delivered thereto.

FIG. 14E is an image of the agar gel illustrated in FIG. 14B after sulforhodamine B dye has been passively delivered thereto.

FIG. 14F is an image of the agar gel illustrated in FIG. 14C after sulforhodamine B dye has been passively delivered thereto.

FIG. 15A is a side view of a grooved microneedle array with channel widths of 200 μm integrated with a microfluidic channel inlet for active drug delivery, in accordance with some aspects of the present disclosure.

FIG. 15B is a perspective view of the grooved microneedle array of FIG. 15A.

FIG. 15C is a cross-sectional view of an active drug delivery microfluidic patch, in accordance with some aspects of the present disclosure.

FIG. 15D is a top view of a microscope image of a 3D printed grooved microneedle array for active drug delivery, in accordance with some aspects of the present disclosure.

FIG. 15E is a perspective view of the microneedle array of FIG. 15D.

FIG. 15F is a left side view of the microneedle array shown of FIG. 15D.

FIG. 16A is an image of a DES gelatin gel before sulforhodamine B dye has been delivered via a grooved microneedle array with channel widths of 200 μm, in accordance with some aspects of the present disclosure.

FIG. 16B is an image of the gel of FIG. 16A undergoing active delivery of sulforhodamine B dye.

FIG. 16C is an image of the gel of FIG. 16A 30 minutes after sulforhodamine B dye has been actively delivered using 100% flowrate for a pump.

FIG. 16D is an image of the gel of FIG. 16A 30 minutes after sulforhodamine B dye has been actively delivered using 50% flowrate for a pump.

FIG. 17 is a schematic diagram of a Bluetooth-integrated drug delivery system, in accordance with some aspects of the present disclosure.

FIG. 18A is an image of a porcine model skin sample after trypan blue stain has been actively delivered via a microneedle array, in accordance with some aspects of the present disclosure.

FIG. 18B is an image of an agarose gel sample after sulforhodamine B dye has been actively delivered via a microneedle array, in accordance with some aspects of the present disclosure.

FIG. 19A is a plot of zeta potential of blank and drug-loaded microneedles, in accordance with some aspects of the present disclosure.

FIG. 19B is a plot of hydrodynamic diameter of blank and drug-loaded microneedles, in accordance with some aspects of the present disclosure.

FIG. 19C is a plot of polydispersity index of blank and drug-loaded microneedles, in accordance with some aspects of the present disclosure.

FIG. 19D is a plot of in vitro release profile of a drug from a drug-loaded microneedle over time, in accordance with some aspects of the present disclosure.

### DETAILED DESCRIPTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a controller device, a process being executed (or executable) by a controller device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other controller devices, or may be included within another component (or system, module, and so on).

In the methods described herein, the steps can be carried out in any order without departing from the principles of the disclosure, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process. The term “substantially” as used herein refers to a majority of, or mostly, as in at least about 50%, at least about 60%, at least about 70%, at least about 80%, at least about 90%, at least about 95%, at least about 96%, at least about 97%, at least about 98%, at least about 99%, at least about 99.5%, at least about 99.9%, at least about 99.99%, or at least about 99.999% or more.

The following discussion is presented to enable a person skilled in the art to make and use aspects of the disclosure. Various modifications to the illustrated configurations or processes will be readily apparent to those skilled in the art, and the generic principles herein can be applied to other aspects and applications within the scope of the present disclosure and the understanding of one of skill based thereon. Thus, the present disclosure is not intended to be limited to particular embodiments or aspects shown, but is to be accorded the widest scope consistent with the principles and features disclosed herein. The following detailed description is to be read with reference to the figures, in which like components or elements in different figures have like reference numerals. The figures, which are not necessarily to scale, depict selected aspects and configurations or processes and are not intended to limit the scope of the disclosure. Skilled artisans will recognize the examples provided herein have many useful alternatives and fall within the scope of the disclosure. In accordance with aspects of the present disclosure, mechanisms (which can, for example, include systems, methods, and media) are provided for using drug delivery systems with grooved microneedles to administer a drug to a region of interest of a subject.

Generally, the present disclosure provides systems, methods, and media for using a drug delivery system comprising at least one microneedle to advantageously place a microneedle patch on a subject's skin and drive or direct a fluid containing a drug solution through a fluid path defined by the at least one microneedle, e.g., a microneedle disposed within a subject's skin. In this way, a drug delivery system can provide transdermal administration of a drug to a region of interest of a subject. Specifically, a microneedle can define a fluid path therein, e.g., a groove extending along a length of the microneedle between a distal tip end and a proximal base end. When the microneedle is applied to the skin of a subject, a drug, e.g., a fluid containing a drug solution, is delivered through the fluid path of the microneedle. More specifically, when a drug-loaded microneedle patch is applied to a surface such as the skin of a subject, a drug solution is released continuously into a region of interest of the subject such that the drug is injected into the skin and is delivered, e.g., to the systemic circulation or desired local tissues. Without wishing to be bound by theory, in some examples, a drug-loaded microneedle patch can provide sustained subcutaneous release of a drug solution over a long period of time, e.g., 24 hours. Thus, one of several features that distinguishes the present disclosure is that the drug solution is provided by microneedles at clinically relevant doses through sustained delivery.

Moreover, using a microneedle-based drug delivery system allows a drug to be provided without delivery by infusion or repeat doses in a short period of time. Correspondingly, first-pass metabolism is avoided and personalization of drug delivery qualities, e.g., dosage, release rate, patch size, and duration of administration, can occur. This confers several key benefits, e.g., the ability to deliver active ingredients by microneedle that hitherto could not be loaded in sufficient amount on a microneedle; the ability to increase drug delivery flexibility, the ability to create microneedle patches that can require shorter application time and/or less frequent reapplication, the ability to enable a sustained drug release profile and facilitate steadier plasmas concentration versus time profile over a longer period, and/or the ability to obtain very high drug concentrations in the vicinity of the microneedle(s), thereby creating a high concentration gradient and further improving drug delivery. Furthermore, the present disclosure optionally allows a drug to be delivered using a microneedle using capillary action of provided by fluid channels in the microneedle, which can beneficially reduce or eliminate the use for external delivery means, e.g., a syringe pump or other pump means, to minimize production cost and increase ease of use of the drug delivery system.

The inventive microneedles also possess remarkable physical characteristics that confer practical benefits. The dimensions of the of the grooves of microneedles are modulated to change the microneedles' drug delivery characteristics, e.g., drug delivery rate. The composition and structure of the microneedle provide excellent penetration into skin as well as structural integrity, e.g., rigidity or semi-rigidity and resistance to breakage.

### Overview of Microneedle Structure and Fabrication

In some aspects, the system includes one or more microneedles, each microneedle comprising a body comprising solid material with a groove, which is disposed within the body and extends along at least a portion of an axial length of the microneedle defined between a pointed apex and a base along a longitudinal axis of the microneedle. Further, the groove defines a fluid channel along a length of the body of the microneedle. While the figures and examples herein illustrate the groove extending along the entire length of the microneedle between the distal pointed apex and the base, thus, defining a longest length of the groove in some aspects, the term “length” is to be construed as a length taken at any point along each respective element of the microneedle.

In some embodiments, the solid material is formed from a flowable material (e.g., a resin that is later cured to form a solid, e.g., a polymer; or a flowable metal, e.g., an alloy that is later cooled to form a solid). In some embodiments, the flowable material is cast onto a mold comprising one or more needle-shaped mold cavities. Solidification of the flowable material in the mold yields the inventive microneedle(s). In some embodiments, the solid material has a hardness of at least 40 Shore A, between 40 Shore A and 100 Shore A, between 60 Shore A and 100 Shore A, between 0 Shore D and 90 Shore D, 10 Shore D and 80 Shore D, 40 Shore D and 80 Shore D, 60 Shore A and 80 Shore D, or at least 80 Shore D.

In some embodiments, the mold comprises an array of needle-shaped mold cavities in a specific geometric configuration. In some embodiments, the flowable material may be a biocompatible resin. For example, dental SG resin may be used. Other suitable types of biocompatible resins include, but are not limited to, BioMed Clear Resin (RS-F2-BMCL-01), Biomed Amber Resin (RS-F2_BMAM-01), Dental LT Clear Resin (RS-F2-DLCL-02), Surgical Guide Resin (RS-F2-SGAM-01), and Dental SG resin (RS-F2-DGOR-01). The biocompatible resin may be photo-curable and, when cured, may yield a hard polymer. In some embodiments, the biocompatible resin or its cured product may include a species selected from chitosan, chitosan polybutylene adipate terephthalate, poly(butylene adipate-co-terephthalate), polyethylene glycol, poly(ethylene glycol) diacrylate, gelatin, gelatin methacyloyl, polyvinyl alcohol, silk, and combinations thereof. Other materials used to fabricate the porous microneedles may include, but are not limited to, polylactic acid (PLA), polyvinyl alcohol (PVA), poly(ethylene glycol diacrylate) (PEGDA), or UV curable polymers.

### Drugs for Use in the Present Disclosure

It will be understood that the drug delivery system discussed herein is compatible with a variety of fluids and/or drugs. In some examples, a drug is a monoclonal antibody, e.g., rituximab, an antibody, a therapeutic peptide, a colony stimulating factor, a low-molecular weight drug, an analgesic, e.g., lidocaine, an anesthetic another drug, or combinations thereof.

In some embodiments, the drug is an analgesic, anesthetic, anti-Alzheimer's, anti-asthma agent, anti-Parkinsonism, antiallergic, antianginal, antiarrhythmic, antiarthritic, antiasthmatic, antibacterial, antibiotic, anticancer, e.g., 5-Fluorouracil, anticoagulant, anti-depressant, antidiabetic, antiemetic, antiepileptic, antifungal, antiglaucoma, anti-gout, antihistamine, antihyperprolactinemia, antihypertensive, anti-inflammatory, anti-migraine, anti-neoplastic, antiobesity, antiparasitic, anti-protozoal, anti-pyretic, antipsoriatic, antipsychotic, antithrombotic, antiulcer, antiviral, anxiolytic, benign prostatic hypertrophy, bronchodilator, calcium hormone or supplement, cardiotonic, cardiovascular agent, chelator, antidote, chemopreventive agent, contraception, diuretic, dopaminergic agent, gastrointestinal agent, gastroprokinetic, hematopoiesis, hemophilia, hormone, hormone replacement therapy, hypnotic, hypocholesterolemic, hypolipidemic, immunomodulator, immunostimulant, immunosuppressant, immunotherapy, lipid regulating agent, male sexual dysfunction medication, multiple sclerosis, muscle relaxant, neuroleptic, nootropic, anti-osteoportic, phytoestrogen, platelet aggregation inhibitor, prostaglandin, radioenhancer for radiotherapy, muscle relaxant, sedative, tranquilizer, and stimulant, respiratory distress syndrome, vasodilator, or vitamin. In some embodiments, a microneedle is provided that is loaded with a vaccine, an electrolyte, an aqueous solution, or another suitable substance.

### Back Substrate

The material attached to the base(s) of one or more microneedles for supportive purposes, e.g., to provide an adhesive backing and/or to position multiple microneedles in an array, is herein referred to as the back substrate. In some embodiments, the back substrate is, or can comprise, a thin elastic. In some embodiments, the back substrate is, or can comprise, a flexible adhesive. In some embodiments, the back substrate is, or can comprise, a woven material. In some embodiments, the back substrate is, or can comprise, a film. In some embodiments, the back substrate is, or can comprise, a bandage or dressing. In some embodiments, the back substrate is, or can comprise, a biodegradable material. In some embodiments the back substrate can act as an intermediate adhesive to a larger patch for clinical application. In some embodiments the back substrate is a continuation of the same porous material comprising the microneedles. In some embodiments the back substrate is a secondary, drug-loaded material, including a drug-loaded porous material.

A polymer that makes a strong bond with the microneedles may be used as a material to form the back substrate. The material used to form the back substrate may be rigid or flexible depending on the application. Suitable flexible materials include, but are not limited to, paper, textile, polyether ether ketone (PEEK), polyethylene terephthalate (PET), polyethylene naphthalate (PEN), polytetrafluoroethylene (PTFE), parylene, and polyimide. Elastic and flexible resins may also be used (e.g., Elastic 50 A Resin, Flexible 80 A Resin). UV curable resins may also be used, such as when there is a need for conformality, flexibility, and elasticity in the microneedle patch. Hard resins may be used for applications having a need for rigid back substrates. A suitable example of a hard resin includes, but is not limited to, Surgical Guide Resin.

In some embodiments, if the back substrate is planar or substantially planar, the “planar area” of the patch is calculated as the area of the patch in the plane defined by the back substrate. In some such embodiments, the “microneedle planar area” of the patch is calculated as the area of a regular polygon, an irregular polygon, a circle, or another suitable shape, wherein the area is defined as the largest area circumscribed by the locus of all lines: (1) in the plane of the back substrate and (2) that connect all microneedles in pairs. Stated more plainly, but without wishing to modify the foregoing geometric definition, the microneedle planar area is the area defined by the perimeter of the microneedles on the patch. In some embodiments, the planar area of the patch is about 0.50, 1.0, 2.0, 3.0, 4.0, 5.0, 6.0, 7.0, 8.0, 9.0, 10, 12, 15, 16, 18, 20, 24, 25, 27, 28, 30, 32, 33, 35, 36,40, 42, 45, 48, 50, 55, 56, 60, 63, 64, 65, 70, 72, 75, 80, 81, 85, 90, 95, 99, 100, 105, 110, 120, 121, 125, 130, 135, 140, 144, 145, 150, 160, 170, 180, 190, 200, 210, 215, 220, or 225 cm². In some embodiments, the planar microneedles area of the patch is about 0.10, 0.20, 0.25, 0.30, 0.40, 0.50, 1.0, 2.0, 3.0, 4.0, 5.0, 6.0, 7.0, 8.0, 9.0, 10, 12, 15, 16, 18, 20, 24, 25, 27, 28, 30, 32, 33, 35, 36,40, 42, 45, 48, 50, 55, 56, 60, 63, 64, 65, 70, 72, 75, 80, 81, 85, 90, 95, 99, 100, 105, 110, 120, 121, 125, 130, 135, 140, 144, 145, 150, 160, 170, 180, 190, 200, 210, 215, 220, or 225 cm². In some embodiments, the planar area of the patch is between about 0.10 and about 1.0, or between about 1.0 and about 5.0, or between about 1.0 and about 10, or between about 5.0 and about 10, or between about 10 and about 20, or between about 10 and about 100, or between about 20 and about 50, or between about 50 and about 100, or between about 100 and about 150, or between about 150 and about 200, or between about or 200 and about 250 cm² . In some embodiments, the planar microneedle area of the patch is between about 0.10 and about 1.0, or between about 0.50 and about 100, or between about 1.0 and about 5.0, or between about 1.0 and about 10, or between about 5.0 and about 10, or between about 10 and about 20, or between about 10 and about 100, or between about 20 and about 50, or between about 50 and about 100, or between about 100 and about 150, or between about 150 and about 200, or between about 200 and about 250 cm².

### Use and Performance of Microneedles

In some examples, the present disclosure provides a system for delivering a drug to a region of interest of a subject using a patch including at least one microneedle. Specifically, at least one microneedle is coupled to a first side of a patch, and a fluid or drug supply, e.g., a reservoir, is coupled to a second side of the patch such that the reservoir and the plurality of grooved microneedles are in fluid communication with one another. In some aspects, the system further includes a micropump contained in the reservoir, and the micropump is configured to direct a drug solution contained in the reservoir through at least one channel in the at least one microneedle. In this way, the drug delivery systems described herein provide a non-invasive, user-friendly, and compact option for delivering a drug to a region of interest in a subject, which in turn can increase subject comfort and satisfaction.

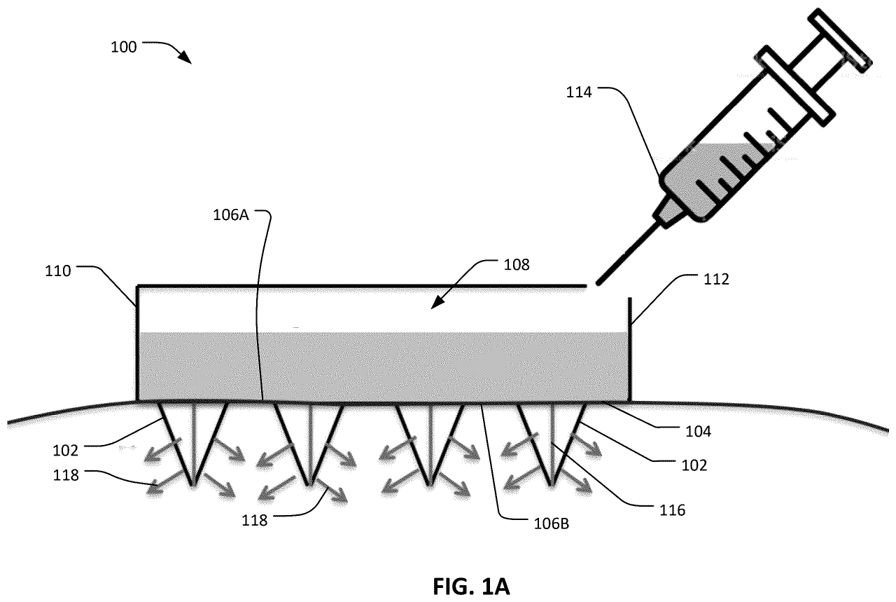

Referring now to FIG. 1A, an example drug delivery system, e.g., a microneedle-based drug delivery system 100, includes a grooved microneedle array 102 and a back substrate or patch 104, and the patch 104 has a first side 106A that defines a bottom wall of a reservoir 108. The reservoir 108 also includes sidewalls 110, 112 to further define the reservoir 108. The grooved microneedle array 102 is coupled to a second side 106B of the patch 104, e.g., a side which is opposite the first side 106A. In other examples, the reservoir 108 is coupled to another side of the patch 104, e.g., a lateral side. Additionally, a syringe pump 114 is used to load the reservoir 108 with a drug and is optionally coupled to the reservoir 108 to allow for active delivery of the drug. Put another way, an operator can actuate the syringe pump 114 to provide positive pressure to the reservoir 108, thus driving the fluid contained therein out through the grooved microneedle array 102 and into a region of interest of a subject. In some aspects, the drug is loaded into the reservoir 108 using different loading techniques, or the reservoir 108 is fabricated with the drug already contained in the reservoir 108, e.g., a drug-coated reservoir, to simplify use of the delivery system 100. In addition to the active pressure provided by the syringe pump 114, capillary action through grooves 116 of the grooved microneedle array 102 causes the drug to flow through the grooves toward a region of interest of a subject, e.g., a transdermal region, as represented by arrows 118.

Referring now to FIG. 1B, another example is illustrated of a drug delivery system 150 that includes a dual reservoir system. In particular, the drug delivery system 150 includes a first reservoir 154 and a second reservoir 156 in place of the reservoir 108 (see FIG. 1A), and a micropump 158 in place of the syringe pump 114 (see FIG. 1A). However, it will be understood that the systems 100, 150 illustrated in FIGS. 1A and 1B, respectively, and the elements thereof are compatible with one another. In the non-limiting example illustrated in FIG. 1B, the first reservoir 154 is coupled to the patch 104, e.g., the first side 106A of the patch 104, and the second reservoir 156. For example, the first reservoir 154 is coupled to a side and/or a bottom of the second reservoir 156, or the first reservoir 154 is disposed within the second reservoir 156. Further, the micropump 158 separates the first and second reservoirs 154, 156 until electrically activated to pump a drug from the first reservoir 154 into the second reservoir 156 so that the drug is delivered through the grooved microneedle array 102. Thus, it will be understood that only the second reservoir 156 is in direct fluid communication with the grooved microneedle array 102, in some examples. Relatedly, a microvalve 160 separates the first and second reservoirs 154, 156, and the micropump 158 and the microvalve 160 are in electronic communication with each other. In this way, the micropump 158 and the microvalve 160 selectively allow fluid communication between the first and second reservoirs 154, 156. For example, the micropump 158 and the microvalve 160 are configured to control a flow rate between the first reservoir 154 and the second reservoir 156 and/or a flow rate of the drug through the grooved microneedle array 102. Thus, the system 150 provides an adjustable drug delivery profile, which advantageously allows the system 150 to be used with a variety of different drugs and subjects.

In some aspects, the micropump 158 is an electrohydrodynamic pump, an electroosmotic pump, a peristaltic pump, or a piezoelectric pump, a vacuum pump, a handheld pump, or another type of pump. In some aspects, the micropump 158 or the microvalve 160 includes an electronic chip that is configured to wirelessly transmit data, e.g., stored data and/or data acquired from a sensor connected to the first and/or second reservoirs 154, 156 to an external device, e.g., by establishing a Bluetooth connection therewith. For example, the electronic chip includes a radiofrequency identification (RFID) tag for identifying and/or tracing a markers in a drug solution. In some other instances, a rate of drug delivery or drug solution outflow of the first and second reservoirs 154, 156 is intermittently checked using the external device. In some aspects, the external device is a smart phone, a wearable electronic device, an RFID tag reader, a laptop, or any other suitable wireless device.

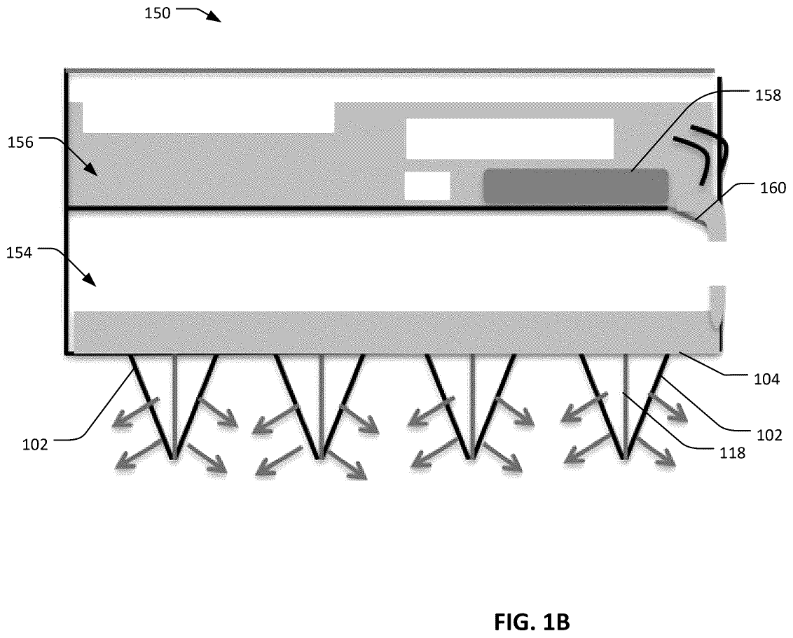

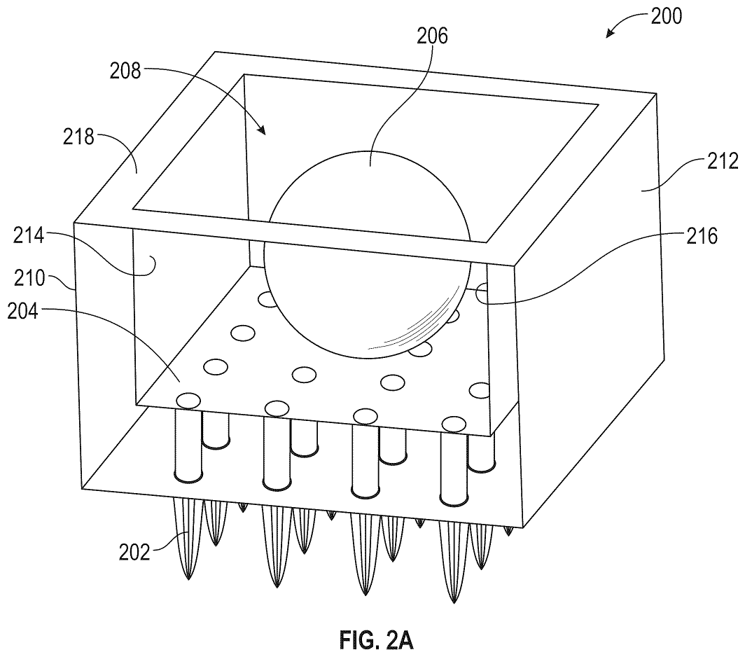

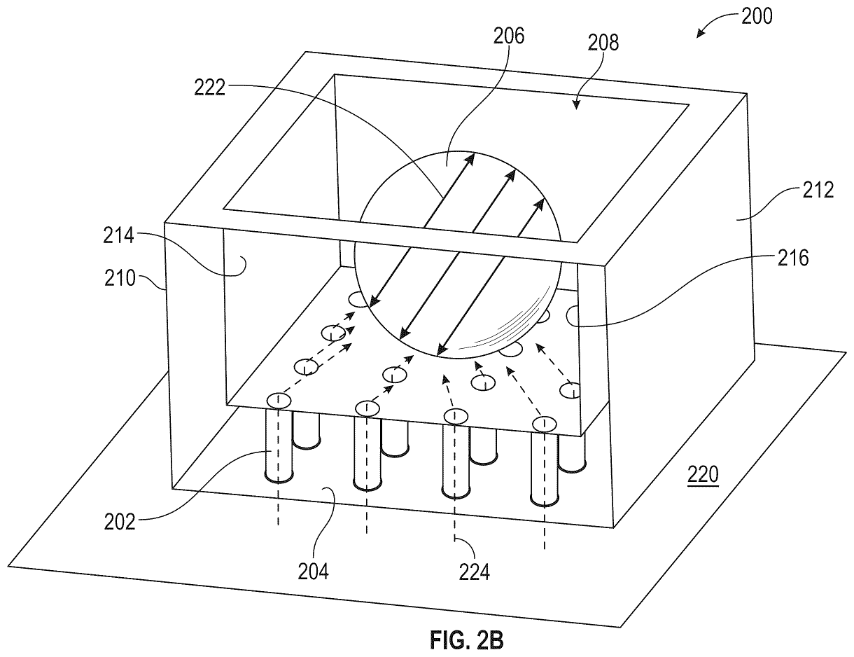

Referring now to FIG. 2A, a perspective view is illustrated of an example microneedle array 200. The grooved microneedle array 200 is configured to deliver a fluid containing a drug solution to an area of interest of a subject via a plurality of microneedles 202, and the microneedle array 200 is coupled to a patch 204. In some examples, the grooved microneedle array 200 is also coupled to an absorbent bead 206 which initially exists in a non-expanded state in a reservoir 208, e.g., a reservoir similar to those discussed above. In some aspects, the reservoir 208 is defined by exterior side walls 210, 212 and the patch 204 that is coupled to the grooved microneedle array 200. The exterior side walls 210, 212 are molded to interior side walls 214, 216 to define a rim 218 of the patch 204. In some aspects, the rim 218 has a width extending between exterior side wall 210 and interior side wall 214 of between 0.50 mm and 5.0 mm, or between 1.0 mm and 4.0 mm, or between 3.0 mm and 2.0 mm, or between 0.25 mm and 1.0 mm, or between 0.50 mm and 0.75 mm. In some aspects, the reservoir 208 is coupled to more than one absorbent bead 206 and extends along a length of the patch 204. In some aspects, the reservoir 208 comprises a biocompatible and photocurable hard resin, e.g., a polymer such as chitosan, chitosan polybutylene adipate terephthalate, or the reservoir 208 comprises a mold that extends from the patch 204 to define a unitary patch construction. However, it will be understood that the absorbent bead 206 may be optional in some examples, meaning that the grooved microneedle array 200 may be in direct fluid communication with the reservoir 208.

In some examples, the grooved microneedle array 200 is configured to sample fluid from an area of interest of subject as well as deliver a drug thereto, as discussed above. Referring now to FIGS. 2B and 2C, the grooved microneedle array 200 extends into a skin model 220 and utilizes capillary action to draw a fluid, e.g., interstitial fluid (ISF), in an upward direction represented by arrows 222 through grooves of the grooved microneedle array 200 which are connected to the absorbent bead 206. In some aspects, the absorbent bead 206 expands in a radially outward direction represented by arrows 222 as fluid is drawn through the grooves of the microneedles 202 and absorbs ISF. As illustrated in FIG. 2B, the absorbent bead 206 expands as it fills, with the ISF drawn in a direction represented by arrows 224 from the grooved microneedle array 200, toward the reservoir 208, and into the absorbent bead 206. In some aspects, the ISF travels through grooves of the grooved microneedle array 200. As illustrated in FIG. 2C, the suction force discontinues once the absorbent bead 206 reaches a fully expanded state, i.e., a filled state, although ISF may continue to travel upward through the grooved microneedle array 200 and into the reservoir 208 via capillary action of the grooves of the grooved microneedle array 200. As previously described, the absorbent bead 206 is removed from the reservoir 208 after reaching a fully expanded state and undergoes downstream analysis, or the absorbent bead 206 remains in the reservoir 208 and is a sensing bead that requires no additional instrumentation to assess properties of the sampled ISF. In some aspects, the reservoir 208 is itself removable from the patch 204 and/or the grooved microneedle array 200, meaning that the reservoir 208 may be removed from the patch 204 to undergo downstream analysis. Thus, in some aspects, the microneedle array 200 also provides a feedback system to monitor the level of a drug in a subject. Specifically, an absorbent bead can indicate if a level of a drug in a subject is within a predetermined threshold, e.g., a “safe” threshold, which an operator can further use to modify or control a drug delivery profile through the system.

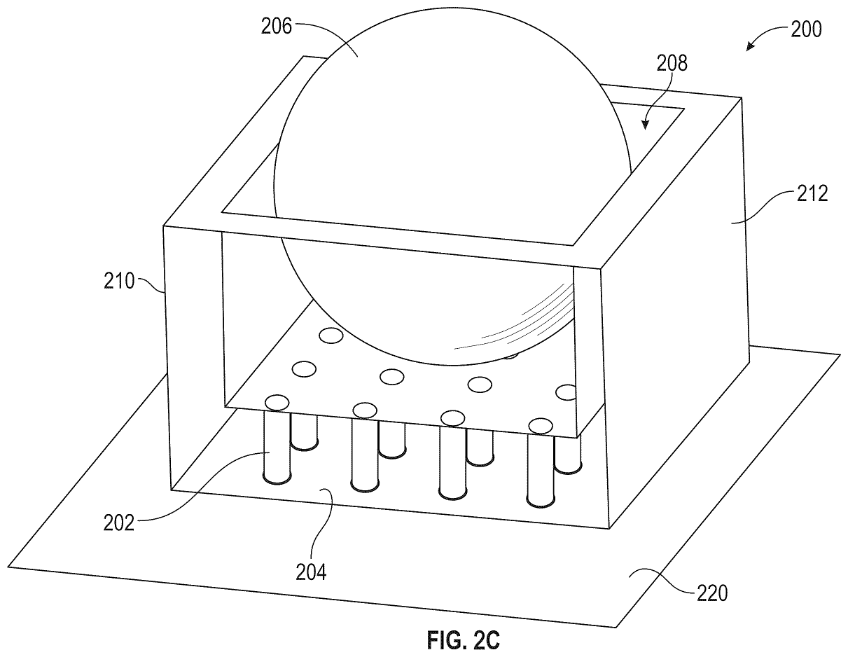

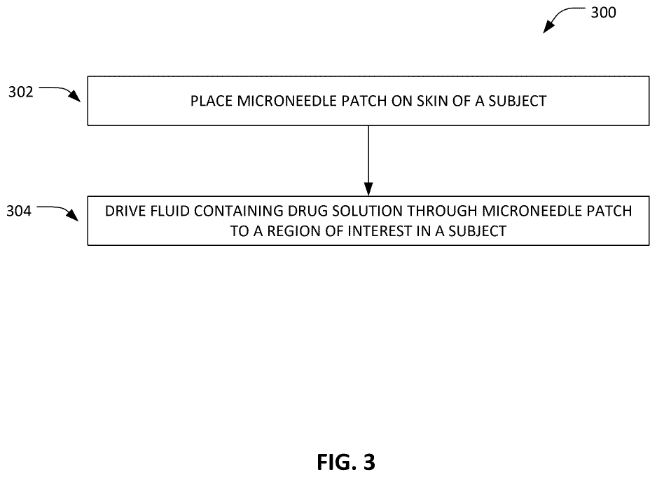

FIG. 3 illustrates a method of delivering a drug to an area of interest of a subject by driving a fluid containing a drug solution through one or more microneedles of a microneedle array and into the area of interest. As discussed above, using a microneedle patch to administer a drug to a subject provides a non-invasive, user-friendly, and compact option compared to traditional drug delivery techniques, e.g., hypodermic needle or infusion. Moreover, using microneedles for drug delivery can also decrease necessary delivery duration while increasing delivery efficacy, as inserting microneedles into a subject's skin is relatively painless in comparison to traditional techniques, which in turn improves subject safety and satisfaction. In some aspects, a method of delivering a drug to a region of interest of a subject includes placing a microneedle patch on the skin of a subject, e.g., on a region of interest of the subject's skin, and driving a fluid containing a drug solution through fluid channels in microneedles of the microneedle patch.

For example, a process 300 of sampling ISF with a microneedle array or patch includes placing the microneedle patch on a subject's skin, e.g., a region of interest of a subject's skin, at step 302. As discussed above, a microneedle patch includes at least one microneedle coupled to a first side of the patch and, in some examples, a reservoir coupled to a second side of the patch. In some aspects, the microneedle includes at least one fluid channel therein, e.g., a central channel and/or an exterior groove, and the fluid channel is in fluid communication with the reservoir. At step 304, the process 300 includes driving a fluid containing a drug solution from the reservoir, through the at least one microneedle, and into the region of interest of the subject. In some aspects, the process 300 includes providing a driving force, e.g., using the pumping mechanisms discussed above, to urge fluid through the fluid channel of the microneedle toward the subject's skin. For example, the driving force is provided by a pump, e.g., a vacuum pump, a syringe pump, a micropump, etc., that is coupled to the reservoir. In other examples, the driving force is provided by compressing a flexible membrane to impart positive pressure on the reservoir. Further, it is contemplated that the suction force is aided by capillary action of the fluid channel of the microneedle, such that the fluid is also passively driven toward the region of interest in the subject, as discussed above.

In some examples, the process 300 further includes controlling a release profile of the fluid containing the drug solution. Specifically, a controller is in electric communication with the pumping mechanism and/or a microvalve coupled to the reservoir, and the controller is used to selectively control the flow rate of the fluid through the microneedles. In some examples, the controller is a wireless controller, e.g., a remote control, a smartphone, a computer-based controller, etc., such that drug delivery is wirelessly controlled. Accordingly, a drug delivery system including a microneedle patch can be used to non-invasively and efficiently deliver a drug solution to an area of interest in a subject. Moreover, the sampling system disclosed herein streamlines offers an easy-to-use option for drug delivery that also enhances subject safety and satisfaction.

### Microneedle Structure

In some aspects, the microneedle includes a conically shaped body defined by an exterior side and extending between a base and an apex point. The base has a center point, and a center line of the microneedle defines a longitudinal axis that intersects the center point of the base and the pointed apex. Further, the base defines an outer diameter of the microneedle. In some examples, a groove or recess is defined in the conically shaped body. The groove is an axial groove that extends substantially parallel with respect to the longitudinal axis of the microneedle, or the groove is offset from the longitudinal axis. In some examples, the groove is not a linear groove, such as a spiral groove that wraps around the body of the microneedle. In some aspects, the groove including a first or right groove face and a second or left groove face. In some aspects, the first or right groove face and the second or left groove face extend between the exterior side of the conically shaped body and the center line. The first or right groove face includes a first or right intersection point with the base, and the second or left groove face includes a second or left intersection point with the circular base. In some aspects, the microneedle includes more than one groove, e.g., two, three, four, five, or more than five grooves.

As discussed above, the microneedle defines a fluid channel therein, and the fluid channel can serve as a pathway along which a fluid containing a drug solution is delivered. In some aspects, the microneedle includes multiple fluid channels, e.g., channels that are in fluid communication with one another. For example the microneedle includes a central channel that extends at least partially along the longitudinal axis. The central channel may also be in fluid communication with apertures defined in the base and/or body of the microneedle. For example, the central channel is in fluid communication with the groove defined in the body, e.g., a groove disposed on the exterior side of the body, such that a fluid pathway comprising the central channel and the groove is formed within the microneedle. In some aspects, the central channel defines a channel length, the groove defines a groove length, and both the channel length and the groove length are measured in a direction that is parallel with respect to the longitudinal axis. In other examples, the microneedle includes two central channels within the body, which may be advantageous for example, to minimize channel blockage during insertion of the microneedle into the skin of a subject.

In some embodiments, the outer diameter, i.e., the diameter of the base, of the microneedle is between 0.20 mm and about 5.0 mm, or between about 0.20 mm and about 4.0 mm, or between about 0.20 mm and about 3.0 mm, or between about 0.20 mm and about 2.0 mm, or between about 0.20 mm and about 1.0 mm, or between about 0.20 mm and about 0.75 mm, or between about 0.20 mm and about 0.50 mm, or between about 0.40 mm and about 0.50 mm, or between about 0.50 mm and about 1.0 mm, or between about 0.80 mm and about 1.2 mm, or between about 1.0 mm and about 2.0 mm, or between 1.0 mm and about 3.0 mm. In some embodiments, a maximum grooved aperture length, i.e., a length that extends between the first or right intersection point and the second or left intersection point, is between 0.10 mm and about 3.0 mm, or between about 0.10 mm and about 2.0 mm, or between about 0.10 mm and about 1.0 mm, or between about 0.10 mm and about 0.75, or between about 0.10 mm and about 0.50 mm, or between about 0.10 mm and about 0.30 mm, or between about 0.20 mm and about 0.30 mm, or between about 0.20 mm and about 0.50 mm, or between about 0.25 mm and about 0.75 mm, or between about 0.25 mm and about 1.0 mm, or between about 0.50 mm and about 0.75 mm, or between about 0.75 mm and about 1.0 mm, or between about 0.50 mm and about 1.0 mm, or between about 0.50 mm and about 1.25 mm, or between 0.75 mm and about 1.5 mm.

In some embodiments, a maximum axial length of the microneedle, i.e., a length that extends between the center point of the base and the apex point, measured in a direction that is parallel with respect to the center line of the microneedle, is between about 0.50 mm and about 10 mm, or between about 0.50 mm and about 8.0 mm, or between about 0.50 mm and about 6.0 mm, or between about 0.50 mm and about 5.0 mm, or between about 0.50 mm and about 4.0 mm, or between about 0.50 mm and about 3.0 mm, or between about 0.50 mm and about 2.0 mm, or between about 0.50 mm and about 1.5 mm, or between about 0.50 mm and about 1.0 mm, or between about 1.0 mm and about 1.5 mm, or between about 0.75 mm and about 1.0 mm, or between about 1.0 mm and about 1.25 mm, or between about 0.80 mm and about 1.2 mm, or between about 0.90 mm and about 1.1 mm, or between about 0.10 mm and about 1.0 mm, or between about 0.10 mm and about 0.75 mm, or between 0.10 mm and about 0.50 mm.

In some embodiments, the channel length of the microneedle is between about 10% and about 100% of the maximum axial length, or between about 50% and about 100% of the maximum axial length, or between about 75% and about 100% of the maximum axial length, or between about 85% and about 95% of the maximum axial length. In some embodiments, the groove length of the microneedle is between about 10% and about 100% of the maximum axial length, or between about 25% and about 75% of the maximum axial length, or between about 40% and about 60% of the maximum axial length, or about 50% of the maximum axial length.

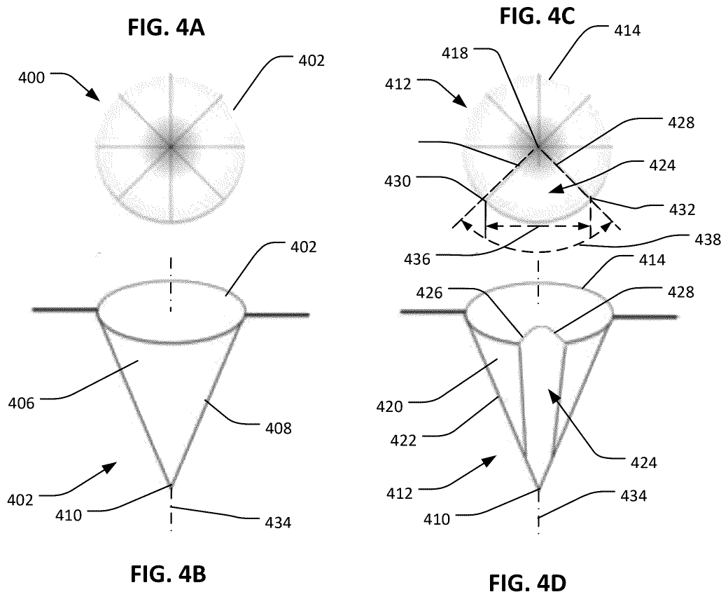

Referring now to FIGS. 4A and 4B, cross-over line (COL) patterns are illustrated that are used to manufacture microneedles via laser etching, according to some aspects of the present disclosure. Specifically, FIGS. 4A and 4B illustrate a symmetric COL pattern 400 of a base 402 of a solid microneedle 404. The symmetric COL pattern 400 results in a completely symmetric and solid body 406 that has an exterior side 408 extending between the base 402 and an apex point 410 along a direction that is parallel with respect to a longitudinal axis 434 of the solid microneedle 404. FIG. 4A illustrates a top view of the symmetric COL pattern 400 overlayed on the base 402, and FIG. 4B illustrates the solid microneedle 404 that is created as a result of using the symmetric COL pattern 400.

FIGS. 4C and 4D illustrate an asymmetric COL pattern 412 of a grooved base 414 to define a grooved microneedle 416. The grooved base 414 has a center point 418. The grooved microneedle 416 includes a body 420 having an exterior surface 422 that extends between the base 402 and the apex point 410 along a direction that is parallel with respect to the longitudinal axis 434 of the grooved microneedle 416. The asymmetric COL pattern 412 includes a groove 424 that is defined by the exterior surface 422, i.e., a recess within the body 420 that extends between a right or first groove face 426 and a left or second groove face 428. In some aspects, a fluid channel for drug delivery is provided in the body 420 through the groove 424. The right or first groove face 426 intersects with the grooved base 414 at a first or right point 430, and the left or second groove face 428 intersects with the grooved base 414 at a second or left point 432. A maximum grooved aperture length is defined as a length extending between the first point 430 and the second point 432, as illustrated by double arrow 436 in FIG. 4C.

In some aspects, the groove 424 extends from the centerline, e.g., the longitudinal axis 434, of the grooved microneedle 416 to the exterior side 408, or the groove 424 does not extend entirely to the longitudinal axis, 434. Put another way, the groove 424 is a surface-level groove, in some examples. In some aspects, the groove 424 defines a groove angle 438, i.e., curved double arrow 438, that is defined as the angle between the first groove face 426 and the second groove face 428 of the groove 424. In some aspects, the groove angle 438 is between about 1 degree and about 150 degrees, or between about 10 degrees and about 130 degrees, or between about 20 degrees and about 110 degrees, or between about 30 degrees and about 90 degrees, or between about 40 degrees and about 70 degrees, or between about 50 degrees and about 60 degrees, or between about 10 degrees and about 50 degrees, or between about 10 degrees and about 25 degrees, or between about 10 degrees and about 20 degrees, or between about 10 degrees and about 15 degrees, or between about 5 degrees and about 10 degrees, or between about 20 degrees and about 30 degrees, or between about 15 degrees and about 20 degrees, or between about 20 degrees and about 25 degrees.

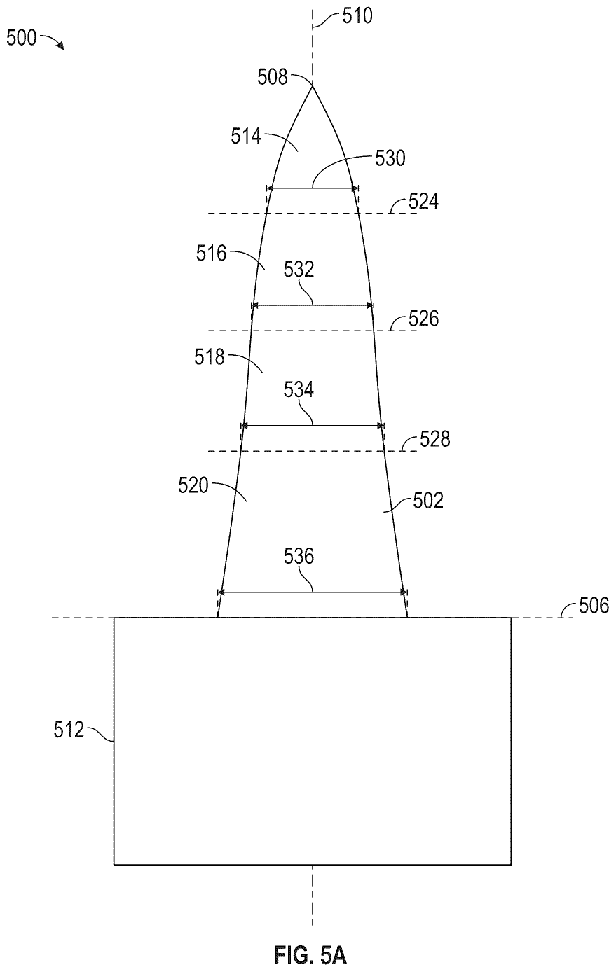

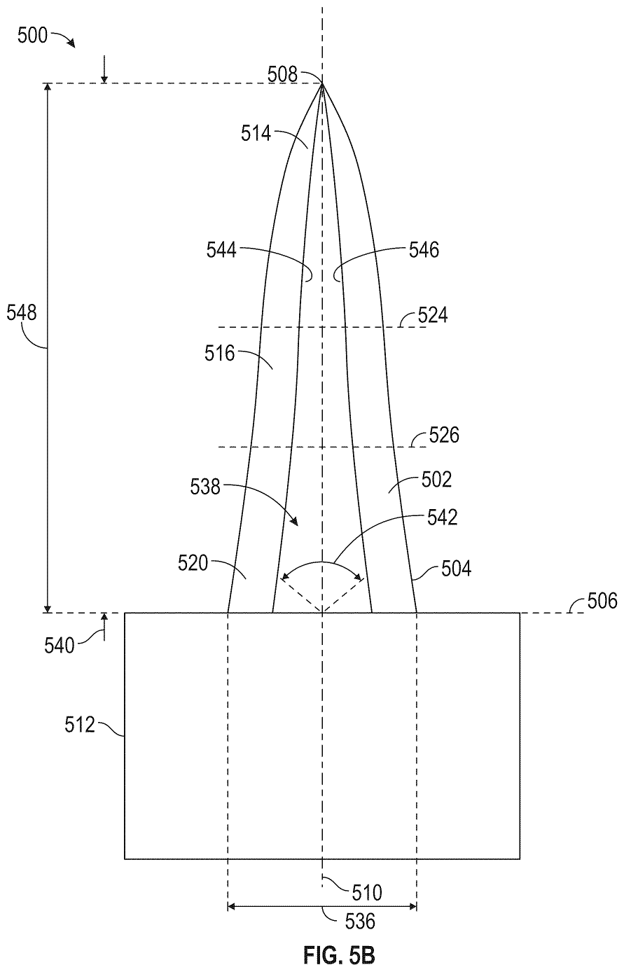

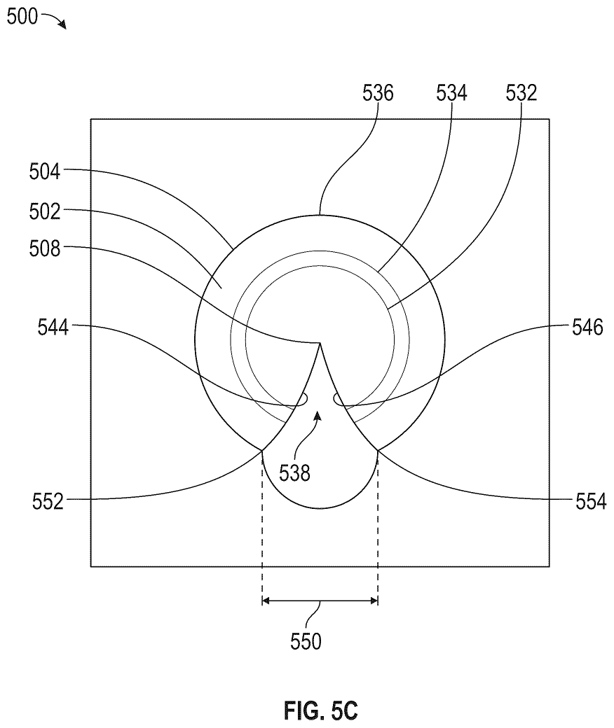

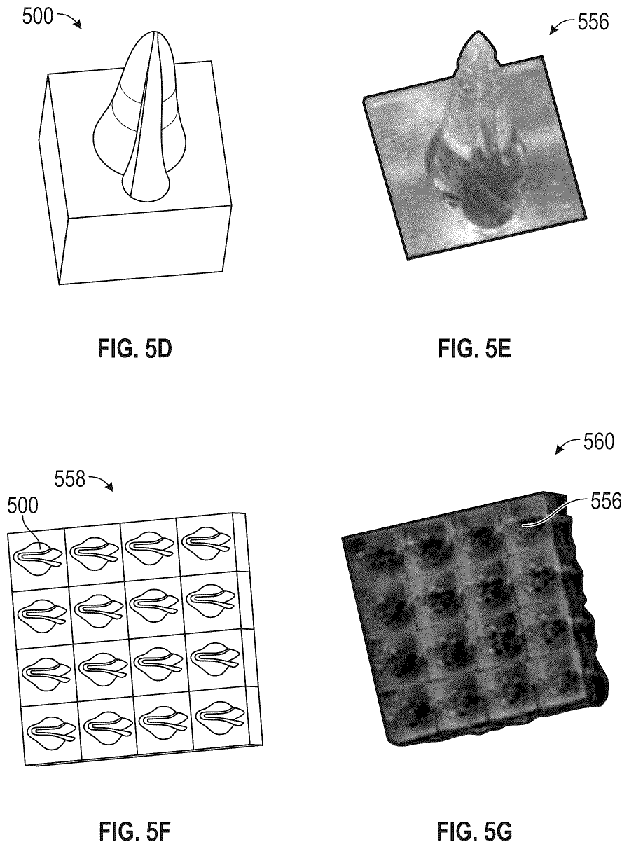

Referring now to FIG. 5A, a rear view of an example grooved microneedle 500 is illustrated. In some embodiments, the grooved microneedle 500 has a body 502 that includes an exterior surface 504. The grooved microneedle 500 extends from a base plane 506 to an apex point 508, and the body 502 is substantially conical in shape. A longitudinal axis 510 of the grooved microneedle 500 extends through the grooved microneedle 500 at the apex point 508 in a direction that is perpendicular with respect to the base plane 506. In some aspects, the grooved microneedle 500 comprises different sections including a tip portion 514, a first intermediate portion 516, a second intermediate portion 518, and a base portion 520, or the grooved microneedle 500 includes only the tip portion 514, the first intermediate portion 516, and the base portion 520. In some aspects, the grooved microneedle 500 includes only one section, e.g., a base portion or a tip portion. The exterior surface 504 extends along the exterior of each section of the microneedle 500. It should be readily understood to one skilled in the art that any combination of possible sections can be used to form the grooved microneedle 500 and that the grooved microneedle 500 may include sections other than those described may exist.

In some aspects, the exterior surface 504 is substantially conical in shape at the tip portion 514 which extends from the apex point 508 to a first transition plane 524. The first transition plane 524 is parallel with respect to the base plane 506 and defines an interface between the tip portion 514 and the first intermediate portion 516. Further, the tip portion 514 has a tip base diameter 530 at the first transition plane 524. In some aspects, the tip base diameter 530 is in a range of between 0.05 mm and 0.50 mm, or between 0.10 mm and 0.40 mm, or between 0.15 mm and 0.3 mm, or between 0.20 mm and 0.25 mm. The exterior surface 504 is substantially convexly curved or sigmoidal in shape at the first intermediate portion 516 which extends from the first transition plane 524 to a second transition plane 526. The second transition plane 526 is parallel with respect to the base plane 506 and defines an interface between the first intermediate portion 516 and the second intermediate portion 518, or between the first intermediate portion 516 and the base portion 520. The first intermediate portion has an outer diameter 532 at the second transition plane 526 that is in a range of between about 0.10 mm and about 0.6 mm, or between about 0.15 mm and about 0.50 mm, or between about 0.20 mm and about 0.40 mm, or between about 0.25 mm and about 0.35 mm, or between about 0.25 mm and about 0.30 mm. In some aspects, the exterior surface 504 is substantially convexly curved, sigmoidal, or concavely curved in shape at the second intermediate portion 518 which extends from the second transition plane 526 to a third transition plane 528. The third transition plane 528 is parallel with respect to the base plane 506 and defines an interface between the second intermediate portion 518 and the base portion 520. The second intermediate portion 518 has an outer diameter 534 at the third transition plane 528 that is in a range of between about 0.10 and about 0.6 mm, or between about 0.15 mm and about 0.50 mm, or between about 0.20 mm and about 0.40 mm, or between about 0.25 mm and about 0.35 mm, or between about 0.30 mm and about 0.35 mm. In some aspects, the exterior surface 504 is substantially convexly curved, sigmoidal, or concavely curved in shape at the base portion 520 which extends from the third transition plane 528 to the base plane 506. Further, the base plane 506 defines an interface between the base portion 520 and a patch or back substrate 512, and the base portion 520 has an outer diameter 536 at the base plane 506 that defines the base diameter of the microneedle, as discussed above. In some aspects, the first intermediate portion 516 interfaces directly with the base portion 520, meaning that the second intermediate portion 518 may be optional.

In some examples, the tip base diameter 530 is between about 25% and about 75% of the outer diameter 536, or between about 50% and about 60% of the outer diameter 536, or about 50% of the outer diameter 536. In some examples, the outer diameter 532 of the first intermediate portion 516 is between about 25% and about 75% of the outer diameter 536 of the base portion 520, or between about 50% and about 75% of the outer diameter 536 of the base portion 520, or about 60% of the outer diameter 536 of the base portion 520. In some examples, the outer diameter 534 of the second intermediate portion 518 is between about 50% and about 100% of the outer diameter 536 of the base portion 520, or between about 60% and about 50% of the outer diameter 536 of the base portion 520, or about 75% of the outer diameter 536 of the base portion 520.

Referring now to FIG. 5B, a front view is illustrated of the example grooved microneedle 500. As shown, the body 502 of the microneedle 500 includes the tip portion 514, first intermediate portion 516, and base portion 520. As discussed above, a microneedle can include a groove or recess therein which can define at least a portion of a fluid channel within the microneedle. In the illustrated non-limiting example, a groove 538 is disposed along the body 502 of the microneedle extends along at least a portion of a maximum axial length 540, i.e., a length that is measured from the base plane 506 to the apex point 508 along the longitudinal axis 510. The groove 538 is formed in the body 502 and defines a fluid channel between the apex point 508 and base plane 506. In some aspects, the groove 538 defines a groove angle 542 that is measured along a radial line (not shown) that extends perpendicularly outward with respect to the longitudinal axis 510. In particular, the groove angle 842 is measured between a first groove wall 544 and a second wall 546 defined by the body 502, the groove angle 542 represented by curved double arrow 542. In some aspects, the groove 538 extends through only the base portion 520 or only the base portion 520 and the first intermediate portion 516 or the base portion 520, first intermediate portion 516, and tip portion 514. In some aspects, the body 502 includes more than one groove and/or a central channel that is in communication with the groove 538, as will be discussed below in greater detail.

In some examples, the groove 538 defines a groove length 548 that is measured in a direction that is parallel with respect to the longitudinal axis 510, and the groove length 548 is between about 1% and about 100% of the maximum axial length 540, or between about 25% and about 100% of the maximum axial length 540, or between about 50% and about 100% of the maximum axial length 540, or between about 75% and about 100% of the maximum axial length 540, or at least about 50% of the maximum axial length 540, or at least about 75% of the maximum axial length 540. Referring now to FIGS. 5A and 5B, the outer diameter 536 of the base portion 520 is between about 25% and about 75% of the maximum axial length 540, or between about 25% and about 50% of the maximum axial length 540, or about 50% of the maximum axial length 540, in some examples. Further, the outer diameter 536 of the base portion 520 is between about 25% and about 75% of the maximum axial length 540, or between about 50% and about 60% of the maximum axial length 540, or about 50% of the maximum axial length 540, in some examples.

FIG. 5C illustrates a top view of the example grooved microneedle 500 including a maximum groove aperture length 550 defined as a length extending between a first point 552 on the first groove wall 544 and a second point 554 on the second wall 546 as represented by arrows 550. In some aspects, the maximum groove aperture length 550 includes length ranges as discussed above. In the illustrated non-limiting example, the groove 538 extends from the exterior surface 504 of the body 502 between the first groove wall 544 and the second wall 546 and to the longitudinal axis 510 (see FIG. 5A). While the first groove wall 544 and second wall 546 are substantially curved in an inward direction toward the groove channel, one skilled in the art would readily understand that the groove walls 544, 546 may include other configurations, e.g., outwardly curved, straight, angled, and/or other configurations. Correspondingly, it is contemplated that the groove 538 may not extend entirely to the longitudinal axis 510 (see FIG. 5A), meaning that the groove 538 may be provided as a surface groove, in some examples.

Referring now to FIG. 5D, a perspective view of the example grooved microneedle 500 is illustrated. Using processes similar to those described herein, the example grooved microneedle 500 is used as a design for manufacturing a molded microneedle 556 as illustrated in FIG. 5E. In some aspects, the molded microneedle 556 is formed using 3D Nanoscribing. In some aspects, the example grooved microneedle 500 is part of a microneedle array 558 as illustrated in FIG. 5F, which is used as a design for manufacturing a molded microneedle array 560 as illustrated in FIG. 5G. While the microneedle arrays 558, 560 of FIGS. 5F and 5G, respectively, are illustrated as square arrays, e.g., 4×4 arrays, it will be understood that microneedles can be arranged in any suitable configuration to define a microneedle array. For example, a microneedle array can be provided as a square array, e.g., a 4×4 grid, a rectangular array, e.g., a 3×4 grid, a circular array, or another suitable configuration, as discussed below.

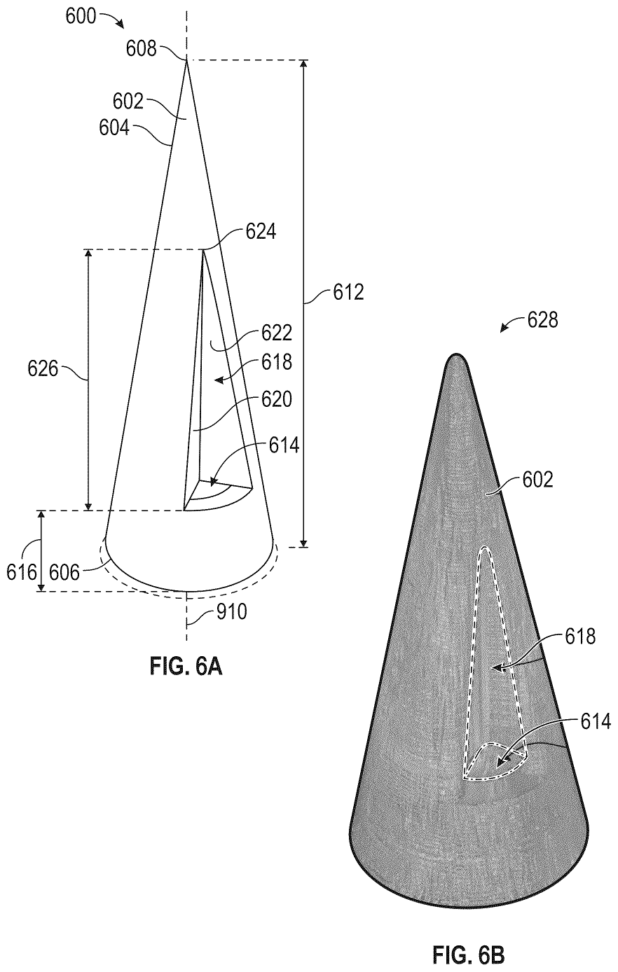

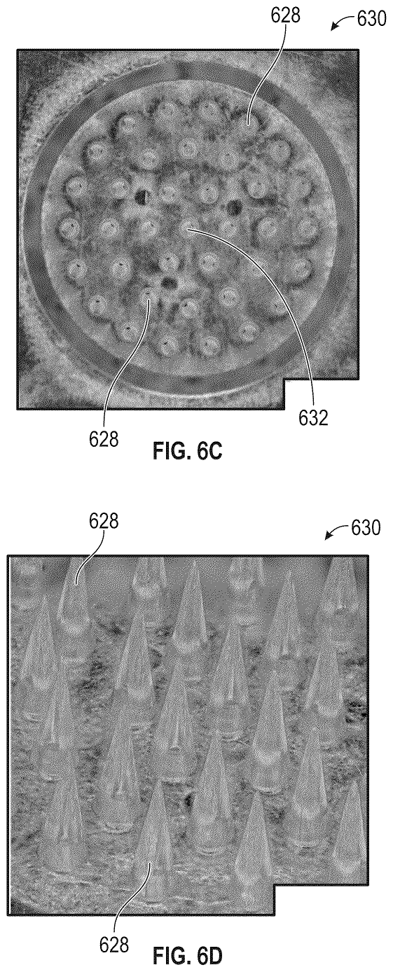

Referring now to FIGS. 6A and 6B, another example is illustrated of a microneedle 600 which includes multiple fluid channels therein. In some aspects, the microneedle 600 is similar to the microneedle 500 illustrated in FIGS. 5A-5C. Referring specifically to FIG. 6A, an isometric view of the microneedle 600 is illustrated, the microneedle 600 having a body 602 that defines an exterior surface 604, a base 606, e.g., a first end, and an apex point 608, e.g., a second end that is opposite the first end. Further, a longitudinal axis 610 extends through the microneedle 600, e.g., through a centerline (not shown) of the microneedle 600 that extends through the base 606 and the apex point 608. The microneedle 600 defines a maximum axial length 612 that extends between the base 606 and the apex point 608 and is measured in a direction that is parallel with respect to the longitudinal axis 610. However, the microneedle 600 differs from the microneedle 500 illustrated in FIGS. 5A-5C in some aspects.

For example, and with continued reference to FIG. 6A, the body 602 of the microneedle 600 is substantially conical in shape, and a central channel 614 is defined within the body 602. Specifically, the central channel 614 is axially aligned with the longitudinal axis 610 and defines a channel length 616 there along, that is, a length measured in a direction that is parallel with respect to the longitudinal axis 610. In some examples, the body 602 includes one or more apertures (not shown) that are defined in the base 606, and the central channel 614 is in fluid communication with the apertures (not shown). As discussed above, a base of a microneedle is generally coupled to a patch and/or reservoir, so including apertures in the base of the microneedle can allow the microneedle to be in fluid communication with the patch and/or reservoir. In the illustrated non-limiting example, one or more apertures can also be defined in the exterior surface 604 of the body 602 so as to provide a path for fluid to flow through the microneedle 600. For example, a groove 618 is defined within the body 602, and the groove 618 is in fluid communication with the central channel 614. That is, the groove 618 defines an opening or aperture in the body 602 of the microneedle 600 which can serve as a site of fluid intake when the microneedle 600 is inserted into a subject's skin to sample ISF. In this way, the central channel 614, the groove 618, and/or any apertures defined in the body 602 and/or base 606 comprise a fluid pathway through which a fluid of a subject can be driven for sampling purposes, e.g., using the pumping mechanisms discussed above.

In some aspects, the groove 618 is a longitudinal groove that is similar in shape to the grooves illustrated in FIGS. 5A-5C, or the groove 618 defines a different shape. In the illustrated non-limiting example of FIG. 6A, the groove 618 defines a substantially triangular or pyramidal shape. Specifically, the groove 618 is defined by a first groove wall 620 and a second groove wall 622, the groove walls 622, 620 defined by the body 602. In some aspects, the groove walls 622, 620 follow the profile of the exterior surface 604 such that the groove walls 622, 620 converge at a groove tip 624. In some aspects, the groove 618 defines a groove length 626 that extends between the central channel 614 and the groove tip 624, measured in a direction that is parallel with respect to the longitudinal axis 610. In some examples, the channel length 616 is between about 5% and about 100% of the maximum axial length 612, or between about 5% and about 25% of the maximum axial length 612, or between about 10% and about 20% of the maximum axial length 612, or about 15% of the maximum axial length 612. In some examples, the groove length 626 is between about 25% and about 100% of the maximum axial length 612, or between about 25% and about 75% of the maximum axial length 612, or about 50% of the maximum axial length 612, as discussed above.

Referring now to FIG. 6B, a perspective view is illustrated of a molded microneedle 628 that is manufactured using the processes described below. For example, the molded microneedle 628 is formed using 3D Nanoscribing based on the design of the grooved microneedle 600 illustrated in FIG. 6A. As illustrated the molded microneedle 628 includes the central channel 614 and the groove 618 that are defined, in part, by the body 602. In some aspects, the molded microneedle 628 is part of a molded microneedle array 630, as illustrated in FIGS. 6C and 6D In particular, FIG. 6C illustrates a top view of the microneedle array 630, and FIG. 6D illustrates a detail view of a portion of the molded microneedle array 630. In some aspects, the microneedle array 630 is a circular array, and the molded microneedles 628 are arranged in concentric circles about a center point 632 of the microneedle array 630. For example, the microneedle array 630 can include one, two, three, four, five, or more than five concentric circles of molded microneedles 628. Four such concentric circles are illustrated in the non-limiting example of FIG. 6C. As discussed above, it will be understood that microneedles can be arranged in any suitable configuration to define a microneedle array. For example, a microneedle array can instead be provided as a square array, e.g., a 4×4 grid, a rectangular array, e.g., 3×4 grid, or another suitable configuration, as discussed herein.

### Manufacture of Microneedles

In some embodiments of the present disclosure, a method of fabricating a grooved microneedle array includes a molding process, such as an additive molding process in which one or more materials are sequentially added to a series of molds. In addition, the methods disclosed herein can include applying one or more surface treatments during the molding process to enhance the material characteristics of the molded products. For example, a method of fabricating a grooved microneedle array includes laser cutting one or more grooved microneedle-shaped depressions into a first material to provide a first mold and casting a second material onto the first mold to fill the depressions with the second material. The method further includes curing the second material to provide a second mold having one or more grooved microneedles formed and removing the cured second mold from the first mold. The method further includes applying a surface treatment, e.g., a plasma surface treatment, to a surface of the cured second mold, applying a release layer to the surface of the cured second mold, casting a third material onto the surface of the second mold, curing the third material to provide the microneedle mold, and removing the microneedle mold from the cured second mold to provide a microneedle patch. Further it will be understood that a variety of fabrication methods can be used to manufacture the microneedles disclosed herein, including, e.g., UV lithography, drawing lithography, deep X-ray lithography, micromilling, deep reactive ion etching (DRIE), wet etch technology, and/or 2D and 3D printing.

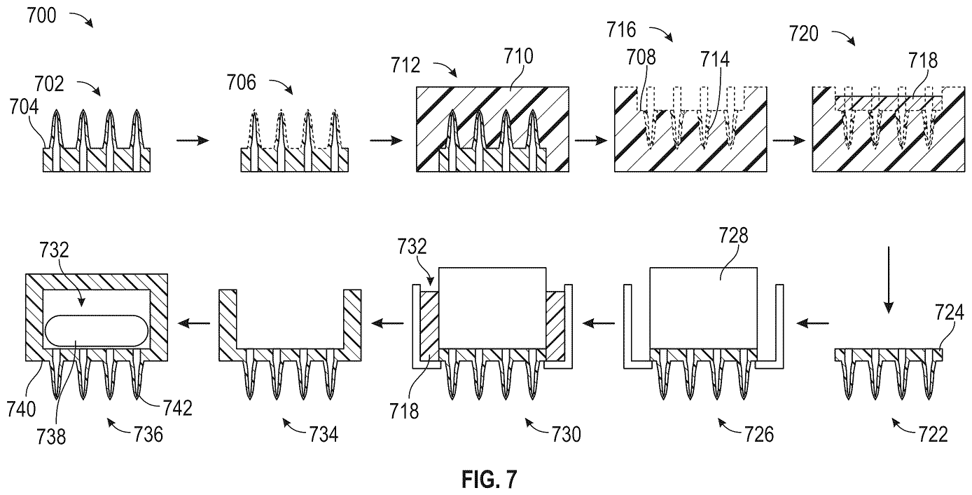

Referring now to FIG. 7, an example process 700 for creating a flexible microneedle patch is illustrated. As shown in process block 702, a microneedle master mold 704 with a high groove aspect ratio is fabricated by using two-photon polymerization that utilizes an acrylic sheet such as an IP-S resin. The master mold is then provided with a surface treatment as shown in process block 706. The microneedle master mold 704 is then used to create a duplicate high-resolution microneedle mold 708 using a moldable microneedle material 710 as shown in process block 712. In some aspects, the moldable microneedle material 710 comprises a silicone elastomer such as a polydimethylsiloxane (PDMS) solution. In other instances, the moldable microneedle material 710 comprises any other suitable moldable material for creating hard microneedles (i.e., microneedles having a Young's modulus significantly higher than that of human skin). After the moldable microneedle material 710 is cast onto the microneedle mold 708, excess moldable microneedle material 710 is removed from the microneedle mold 708, such that the moldable microneedle material 710 fills only microneedle-forming cavities 714 of the microneedle mold 708 as shown in process block 716.

After the excess moldable microneedle material 710 is removed, with the microneedle-forming cavities 714 being filled with the moldable microneedle material 710, a biocompatible and photocurable hard resin 718 is added and allowed to cure onto the microneedle mold 708 after being placed in a vacuum chamber to remove bubbles from the liquid state PDMS, as shown in process block 720. In process block 722, the resulting PDMS mold 724 is peeled from the microneedle mold 708 to form grooved microneedle forming cavities and treated with oxygen plasma before being salinized. Salinization prevents the PDMS mold 724 from sticking to the casting pre-polymer making it easily detachable. In process block 726, backside reservoir walls 728 are fabricated by placing an outer ring mold and a reservoir mold around the PDMS mold 724 using ECOFLEX structures. In process block 730, the biocompatible and photocurable hard resin 718 is deposited in a reservoir cavity 732 defined between the reservoir walls 728. The PDMS mold 724 with the ECOFLEX structures is then placed in a vacuum and is allowed to cure in process block 734 before the ECOFLEX structures are removed. In process block 736, an absorbent bead 738 is inserted into the reservoir cavity 732 which is then sealed using a biocompatible and photocurable hard resin molding method similar to that previously described. A resultant flexible microneedle patch 740 comprising a final mold structure including the reservoir cavity 732, grooved microneedles 742, and the absorbent bead 738, is then used for transdermal sampling of ISF.

The flexibility of the flexible microneedle patch 740 allows the flexible microneedle patch 740 to readily conform to any portion of the human body (e.g., arm, knee, neck, etc.), while the hardness of the grooved microneedles 742 allows them to effectively penetrate the skin of a subject to permeate the skin for drug administration. Further, using the above-described method (see FIG. 7), flexible microneedle patches of varying sizing and including varying numbers of microneedles can be created. Accordingly, sampling and sizing can be adjusted accordingly for a given application.

In some aspects, the absorbent bead 738 is omitted from the system and a drug or drug solution is instead deposited in the reservoir cavity 732. In some aspects, the drug is loaded into the reservoir cavity directly before use instead of during fabrication. The flexibility of the microneedle patch 740 allows the flexible microneedle patch 740 to readily conform to any portion of the human body, e.g., arm, knee, neck, etc., while the hardness of the grooved microneedles 742 allows them to effectively penetrate the skin of a subject to permeate the skin for drug administration. Further, using the above-described method, flexible microneedle patches of varying sizing and including varying numbers of microneedles is created. Accordingly, dosage and sizing are adjusted for a given application.

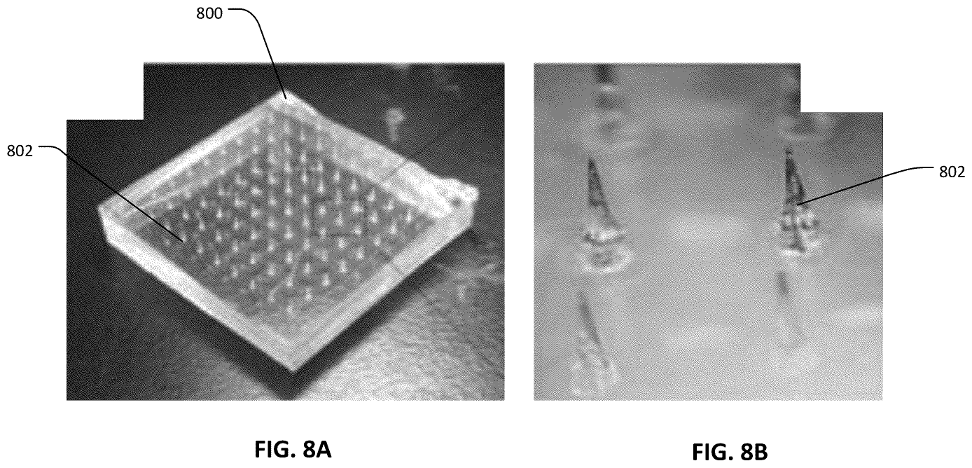

Referring now to FIG. 8A, a PVA microneedle patch 800 is illustrated. The patch 800 includes fine microneedles 802 that are fabricated to produce various tip sizes by adjusting number of lines and writing speed settings during fabrication. In some aspects, techniques and methods such as those described above for FIG. 7 are used to fabricate the microneedles. A microscopic image of individual fine microneedles 802 is illustrated in FIG. 8B.

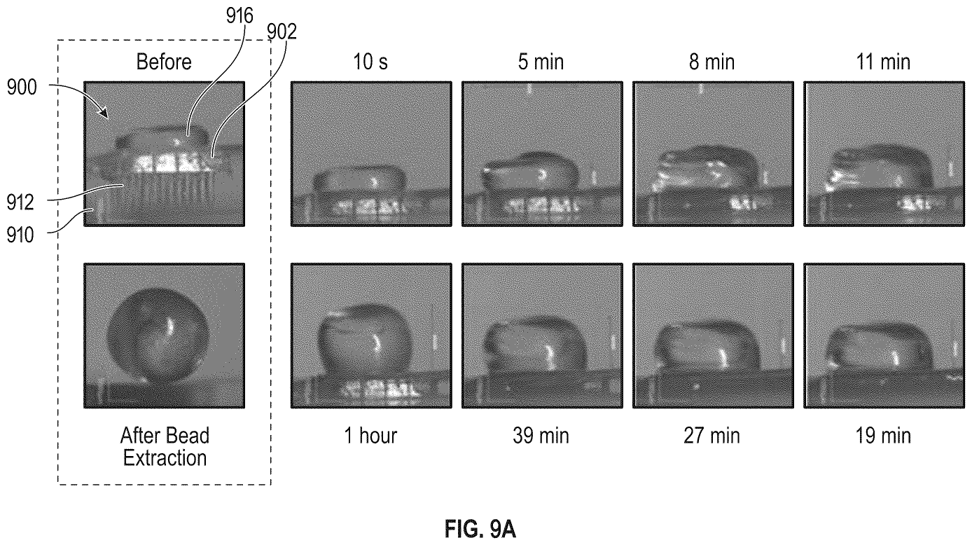

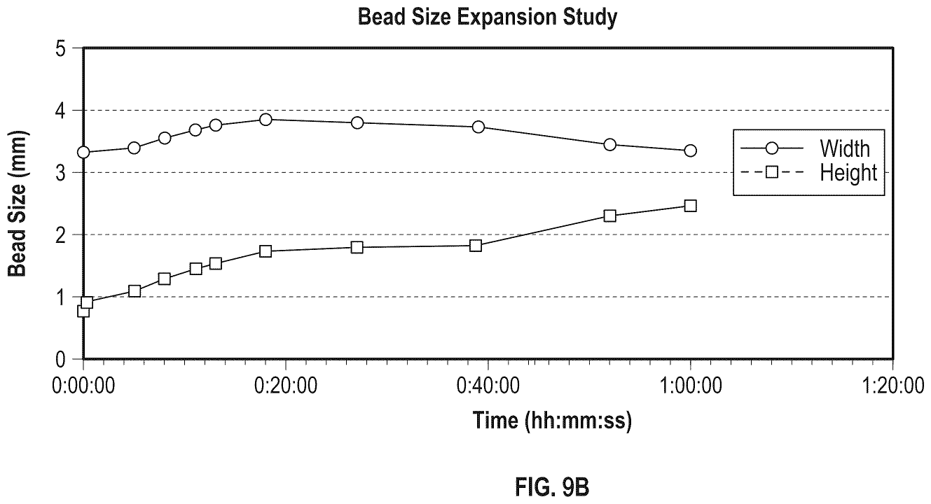

Referring now to FIG. 9A, sampling kinetics of a grooved microneedle patch 900 are illustrated. The patch 900 includes a bead 902 connected to a back substrate 904 of the patch 900. Grooved microneedles 906 are disposed on an opposite side of the back substrate 904 relative to the bead 902 and are applied to a skin model 908 made from agarose phantom gel. For easy visualization, the skin model 908 is loaded with Rhodamine B dye, which is sampled into the bead 902 through the grooved microneedles 906. The sequential images in FIG. 9A illustrate the expansion of the bead 902 to validate the fluid transport characteristics of the grooved microneedle patch 900. The bead 902 expands as the dye is sampled through the grooved microneedles 906. Data of the bead's size increment versus time is collected and analyzed as shown in FIG. 9B, proving the workability of the grooved microneedle patch 900.

### EXAMPLES

### Example 1

### Introduction

The major obstacle in transdermal delivery of medications is the lack of technology to provide accurate, sustained delivery at clinically relevant doses. The present example describes a transdermal microneedle patch that was developed to carry a drug payload sufficient to deliver a medically effective dose. This disclosure proposes to use cutting edge, low-cost drug technology to deliver sustained release of ketamine and its metabolite (2R,6R)-HNK and to validate the technology in a proof of principle study by demonstrating the effectiveness of ketamine and (2R,6R)-HNK transdermal delivery in rats exposed to chronic pain and stress provocation. Clinical trials have shown that racemic ketamine is effective at producing prolonged effects on chronic pain and depression following repeated infusions. More recently, S-ketamine delivered intranasally has been approved by the Food and Drug Administration (FDA) to treat major depressive disorder and suicidal ideation. However, both repeated infusion and intranasal drug administration routes have limitations. Medication delivery by infusion requires dedicated space and manpower for managing an intravenous line in patients during the repeated infusions. Although intranasal administration has advantages, the low intrinsic permeability of hydrophilic medications, the rapid mucociliary clearance and enzyme degradation limits drug absorption. In the case of ketamine, repeated doses are required during each treatment period. Due to the psychotomimetic effects of the drug, most patients will need assistance with drug administration to achieve the optimal dose. By contrast, sustained subcutaneous release of medications is a more flexible option that can accomplish the same medical objectives while enhancing patient compliance, reducing costs, and increasing access to treatment. In addition, this technology can deliver a drug for up to 24 hours, offering a viable system for drug delivery for prehospital trauma care.

### Pain and PTSD

Chronic pain impacts the lives of 20-30% of U.S. adults. The incidence of pain conditions is significantly greater in military service members and veterans, given the likelihood of injury during combat and training. The economic burden, in direct medical costs, lost productivity, and disability programs, associated with chronic pain is approximately $560 billion yearly. Although opioids may be effective in the early injury period, their utility for chronic pain conditions is limited and, in many cases, opioids can lead to greater hyperalgesia, tolerance and dependence. Thus, new pharmacotherapies are urgently required to alleviate chronic pain conditions. hyperalgesia, tolerance and dependence.

World Mental Health Surveys on Trauma and PTSD indicate that only 25-40% of PTSD cases resolve within 1 year. For the majority of subjects, the mean duration of symptoms equated to 6 years across a range of trauma types. Worryingly, combat-related PTSD was associated with a mean duration of 13 years. This debilitating disorder can be triggered by exposure to an actual or threatened death, serious injury, or sexual violence. PTSD is a significant medical, social, and economic burden, which substantially impacts combat readiness and occupational functioning. Exposure therapy and cognitive behavioral therapy are utilized as the first line of treatment for PTSD. However, such therapies can be difficult to administer effectively during deployment. Moreover, 33% of individuals within the general population are deemed treatment resistant. This treatment resistant population comprises patients that fail to respond to cognitive behavioral therapy (50% of all subjects assigned to this treatment strategy) and 40% of all subjects receiving pharmacotherapy with selective serotonin reuptake inhibitors (SSRIs).

Clinical studies have established that infusion of subanesthetic doses of racemic ketamine provides support for opioid analgesia during postoperative pain, effectively reducing the amount of opioids required to diminish pain. Racemic ketamine can also produce sustained analgesic effects when administered repeatedly by infusion for several chronic pain conditions. Treatment for spinal cord injury pain requires ketamine infusions of 0.42 mg/kg/h to 0.4 mg/kg ranging from 17 minutes to 5 hours for 7 consecutive days. For complex regional pain syndrome (CRPS), ketamine infusions of 2 mg/h for 4 days or 0.35 mg/kg/h over 4 h daily for 10 days have demonstrated efficacy and long-lasting relief from pain. These brief but repeated ketamine treatment regimens produced protracted analgesic effects lasting for weeks. Reports also suggest a potential indication for ketamine as a treatment for PTSD. Retrospective assessment determined that racemic ketamine administered intraoperative mitigated the subsequent development of PTSD in burned service members. Although one study described the potential of S-ketamine to exacerbate PTSD symptoms after treatment, racemic ketamine did not potentiate symptoms when administered during the peritrauma period. In a randomized control trial, low-dose ketamine was shown to alleviate symptoms of PTSD within hours of infusion, relative to placebo (midazolam) treated controls. The rapid resolution of PTSD symptoms in adults and children following a single dose of ketamine persisted for over one week before the re-emergence of symptoms. Furthermore, repeated administration of racemic ketamine (6 times over a 2-week period, 0.5 mg/kg 40-min infusions) produced persistent remission in treatment resistant subjects starting as soon as 24 h following the first infusion. After the cessation of treatment, the median time to relapse was 41 days. Similarly, in a cohort of treatment-resistant depressed patients with comorbid PTSD, sublingual ketamine twice weekly effectively reduced the number of inpatient days spent in hospital. More recently, a marked effect of high dose ketamine on PTSD symptomology has been reported. In that study, six 1-hour infusions of ketamine (mean final dose was 163.83 mg (SD=52.50)) reduced the patient health questionnaire scores from 18.5 to 9.5 and PTSD checklist for DSM-5 from 56.2 to 31.3. Research conducted with hundreds of patients has demonstrated the unique ability of low-dose racemic ketamine to rapidly and robustly alleviate suicidal ideation in patients with mood disorders. The rapid resolution of suicidal ideation by ketamine is unique among psychoactive medications. Despite the transformative pattern of results for ketamine in military medicine, ketamine's widespread use for analgesia has been hindered by concern for side effects, such as unpleasant psychotomimetic symptoms, sedative effects and potential abuse liability.

### (2R,6R)-HNK

Recent preclinical studies and pilot data provided have drawn attention to the value of the ketamine metabolite 2R,6R-hydroxynorketamine ((2R,6R)-HNK)) in assays relevant to mental health endpoints and pain conditions in humans. (2R,6R)-HNK is the secondary metabolite obtained from the metabolism of racemic ketamine, or the R-isomer of ketamine, but not from S-ketamine. Unlike ketamine, (2R,6R)-HNK does not produce the dissociative effects, inherent rewarding properties, ataxia, or acute amnesia associated with N-methyl-D-aspartate (NMDA) receptor antagonism. (2R, 6R)-HINK retains the beneficial behavioral profile of ketamine on endpoints relative to mood making it an ideal drug development candidate for PTSD. Evidence supporting the potential antinociceptive effects of (2R, 6R)-HNK are new but promising. Antinociceptive effects of (2R,6R)-HNK were first reported in mice with a spinal nerve injury 24 hours following a single injection of (2R,6R)-HNK26. Utilizing a rodent model for complex regional pain syndrome (CRPS) and postoperative pain, the analgesic effects of (2R,6R)-HNK persisted up to five days following administration. The pilot data replicated the long-lasting antinociceptive effects of (2R,6R)-HNK in rodents on thermal nociception. Moreover, the analgesia was mediated by glutamatergic α-amino-3-hydroxy-5-methyl-4-isoxazolepropionic acid receptors (AMPARs) and not by opioid receptors. Antinociception was restricted to the (2R,6R)-HNK isomer and not extended to (2S,6S)-HNK, the metabolite obtained from S-ketamine. Thus, (2R,6R)-HNK is a novel non-opioid based compound of interest for development as a therapeutic for chronic pain conditions and PTSD.

The proposed transdermal microneedle array (MNA) delivery device is a minimally invasive closed delivery system and applicable for use in the home setting or at the battlefront. Moreover, (2R,6R)-HNK loaded MNA patches represent a minimal resource burden relative to ketamine infusion. The transdermal MNA-based delivery device avoids first-pass metabolism, provides flexibility in dosage, release rate, patch size, and duration of application to allow for optimization of medical care. Here, microneedle innovations are utilized to improve drug loading capacity to enable a sustained delivery release profile, and therefore, facilitate steadier plasma concentration versus time profile over a longer period. Additionally, the device improves convenience, which has a positive benefit of increased compliance. There is a strong rationale supporting the feasibility of using (2R,6R)-HNK for PTSD and pain endpoints. Not only are these studies warranted, but the range of dose, duration and need for repeated dosing for these different endpoints illustrates the flexibility and unique capability of the MNA patches to deliver medications effectively. The (2R,6R)-HNK data is contrasted with those of MNA delivered ketamine at an infusion rate known to produce the desired behavioral outcomes. This ensures that the beneficial effects of (2R,6R)-HNK are comparable to that of ketamine at this preclinical stage. The MNA patches are patient specific and programmable by the physician for dosage and release rate. Overall, the development of this cutting-edge device for military medicine is an innovative approach that will help treat active service members and veterans more easily for chronic pain conditions and PTSD. The device is also useful for field use and combat casualty care.

### Materials and Methods

The research tasks were to address the primary challenge of making flexible conformal MNA patches with a substrate that is flexible and conformable with microneedles that are hard enough to penetrate the skin reliably, and to fabricate grooves on the microneedle that provides microfluidic routes for drug transport from the reservoir to the dermis layer of skin.

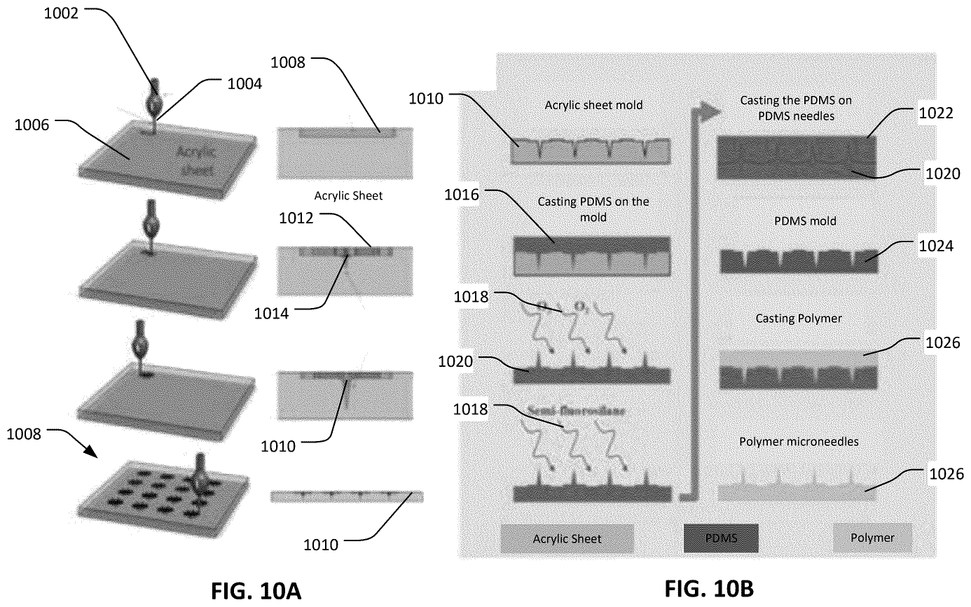

Molds for MNA were made by using the so-called cross-over lines (COL) method, including using asymmetric crossover line patterns, as illustrated in FIG. 10A. In previous approaches, a drug was loaded into the tip of a MN by casting drug-loaded prepolymer solution in the mold, as illustrated in FIG. 10B. However, there was an inherent upper limit to the drug to polymer ratio to ensure mechanical hardness and stability. Large quantities of drug delivery, e.g., in tens of mg, would require fabrication of large area patches which may be impractical for small animals. Moreover, not all drugs can sustain high temperatures used during the curing process. The present example proposed using a separate large capacity drug reservoir and a COL method to fabricate hard microneedles using biocompatible hard dental resin with grooves. The grooves served as a microfluidic channel to transport a drug from the reservoir transdermally to the skin. Grooves were fabricated simply by changing the COL pattern during fabrication of the mold process, as illustrated in FIG. 10A, as well as adjusting the laser intensity and laser writing speeds. A laser cutter 1002 was controlled by an external control system to direct a laser beam 1004 to engrave an acrylic sheet 1006 in a specified pattern 1008. During fabrication of a microneedle-forming mold 1010, the laser cutter 1002 was used to cut or engrave cross lines 1012 that overlap only at their center cross point 1014. As the laser cutter 1002 passes the center cross point 1014 with each cross line 1012, a pointed cone took shape at the center cross point 1014, thereby forming the microneedle-forming mold 1010 from a microneedle forming cavity.

Referring now to FIG. 10B, a first moldable material 1016 was cast onto the microneedle forming mold 1010 and allowed to cure before being extruded from the microneedle forming mold 1010. The first moldable material 1016 as treated with plasma 1018, i.e., oxygen plasma, air plasma, nitrogen plasma, or another type of plasma, to form a protrusion mold 1020. A second moldable material 1022, i.e., PDMS, a chitosan solution, comprise polyethylene glycol (PEG), poly(ethylene glycol) diacrylate (PEGDA), gelatin, gelatin methacryloyl (GeIMA), polyvinyl alcohol (PVA), silk, or any other suitable material, was cast onto the protrusion mold 1020. The second moldable material 1022 was then allowed to dry, thereby creating a hollow microneedle array 1024 including a plurality of grooved microneedles 1026. Similarly, the plurality of grooved microneedles 1026 may correspond in size and shape to the plurality of grooved microneedles 1026 of the microneedle protrusion mold 1020.

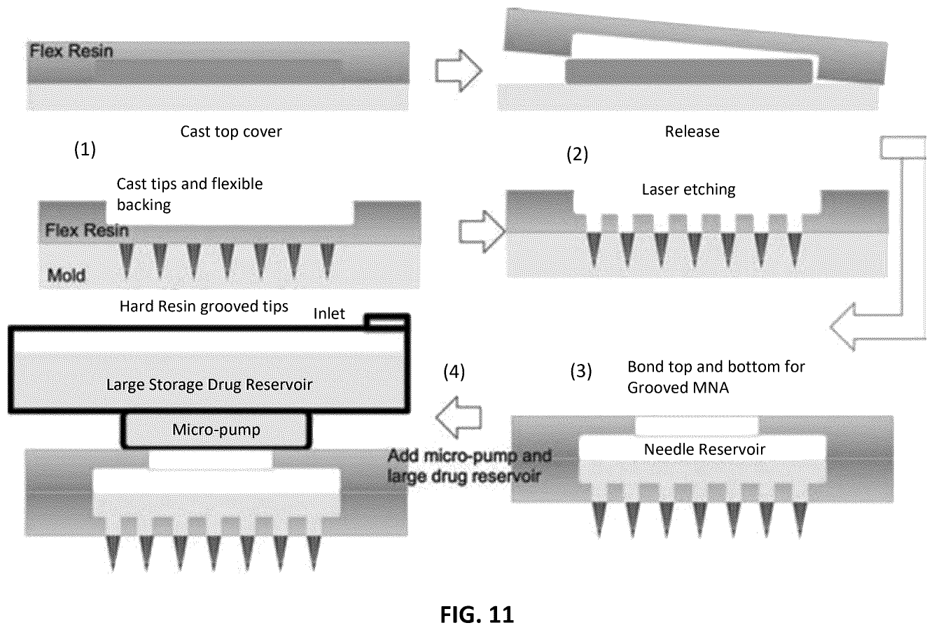

Referring now to FIGS. 4A-4D, different COL patterns and views are shown. Specifically, FIGS. 4A and 4B illustrate top and side perspective views, respectively, of a symmetric COL laser engraving pattern that resulted in a conical MN tip, and FIGS. 4C and 4D illustrate top and side perspective views, respectively, of an asymmetric COL laser engraving pattern that resulted in a grooved MN tip. In some aspects, the grooves were made from a hard biocompatible dental resin, and the backing was made of an elastic flexible resin. This addresses the challenge of making a conformable MNA with hard tips. A separate cavity was also molded to serve as a top to form the microneedle drug reservoir, as illustrated in FIG. 11. Additionally, FIG. 11 illustrates the laser etching and casting of a grooved MNA and a local needle drug reservoir to create a passive drug delivery system. The system further included a micro-pump and large storage drug reservoir, as previously discussed, according to some aspects. Since the drug was not loaded onto the tips of the microneedles, hardness of the microneedles was maintained to achieve large drug dosage.

Delivery rate of the drug was found to be dependent upon the flow rate through the groove as a result of capillary action; it was estimated to be in the range of 1~100 μL/min. Altering the dimensions of the groove controlled the exact rate of drug release, e.g., narrower channels release the drug slower than wider channels. More precise control was achieved through the addition of the micropump that is activated electronically, as illustrated in FIG. 11.

### Franz Cell Ex-Vivo Setup and Characterization

It is advantageous to develop a good representation of a skin sample in order to assess the transdermal release profile of (2R,6R)-HNK and ketamine ex-vivo using a Franz cell. The setup of the Franz cell included a top chamber and a bottom chamber where porcine skin is sandwiched between them. To mimic body temperature, water at 37 degrees F. was pumped into the bottom chamber. Before performing the release study, each skin sample was evaluated to ensure the skin barrier is undamaged which was achieved measuring transepithelial/transendothelial electrical resistance (TEER) across the skin in the Franz cell using an ohm-meter. An undamaged skin sample had a TEER value of approximately 60 KΩ/cm². Next, drug permeation through a full thickness human skin sample was gauged through infusion of 1 mL of concentrate Ketamine/HNK solution for infusion to be added to the top chamber. The bottom chamber was filled with phosphate buffer solution (PBS) and the permeation of the drug was then evaluated for 24 hours. Overall, these testing methods facilitated calibration of the setup, reveal various factors affecting the drug release profile, and provided a useful comparison between topical and transdermal drug delivery.

### Ex-Vivo Validation of Drug Delivery

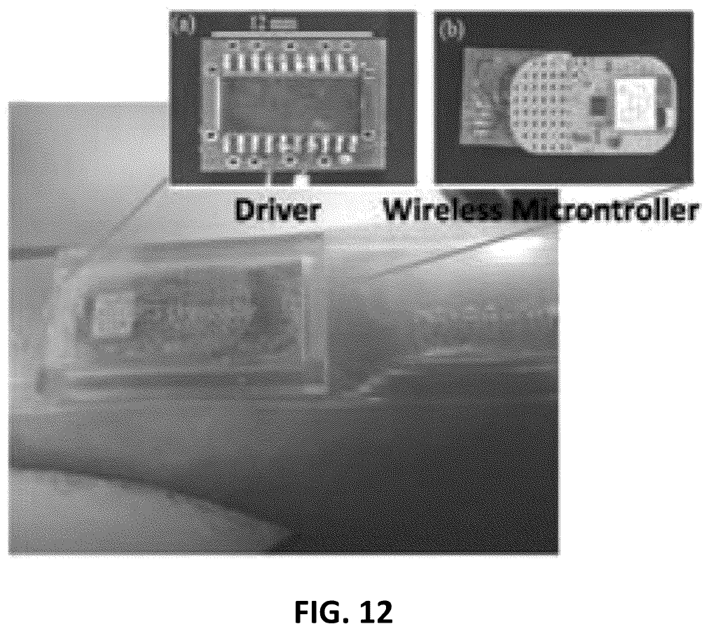

In some aspects, the drug delivery device illustrated in FIG. 11 was modified to provide more active control, e.g., turning the device on or off, regulating the delivery rate on-demand, through inclusion of a miniaturized electro-osmotic pump (EBP Series, Takasago microfluidics) that can generate 1 MPa pressure, and flow rate is controlled precisely with DC voltage bias, e.g., +1 μL/min for each 2.4V increment. A larger drug reservoir, e.g., a drug reservoir with a volume of approximately 50-100 mL, held a sufficient quantity of the drug for a multi-day treatment. A miniaturized, re-usable electronic board including a driver, microcontroller, and wireless circuitry was connected to the grooved MNA patch via flexible ribbon connectors. The electronic or wireless circuitry for active drug delivery, as illustrated in FIG. 12, was used in conjunction with a Bluetooth connected smartphone app or a computer, which the electronic board to be programed to activate the micropump at a set time or duration. In some aspects, wireless circuity was used in conjunction with Wi-Fi, Bluetooth, or another radio wireless communication standard. Adjusting the voltage applied can further be used to control the flow rate of the device. The circuit board was fabricated and subjected to electrical characterization to assess precision in timing, and to ensure adequate driving voltage or power needed to drive the micro-pump exists. Additionally, the micro-pump was independently characterized for flow rate and for the relationship encoded into any software used.

### Characterization of Active Drug Delivery in Ex-Vivo Setup

To evaluate the transdermal release profile of HNK from the grooved microneedle patch, a 5×5 grooved MN array patch occupying a 0.25×0.25 cm² area was fabricated and applied on porcine skin in a setup similar to the previously described Franz cell setup. The ex-vivo skin model was in contact with the reservoir containing PBS which served as artificial interstitial fluid. Skin models of different thicknesses were explored, and (2R,6R)-HNK and ketamine release profiles from the microneedles penetrating the excised skin sample into the reservoir were assessed using UV-Vis spectroscopy. Each compound had a characteristic peak in the UV-Vis spectrum, e.g., ketamine has two absorbance peaks at 203 nm and 263 nm. The cumulative release profile of ketamine/HNK in the bottom reservoir from the needles was measured using a UV-Vis spectrum analyzer.

To assess the functionality of the release drug, an ELISA test (Neogen Corporation) was used. To demonstrate control over the release profile, the ex-vivo test discussed above was performed with pulsed cyclic and stepped profiles with different parameters. Specifically, pulsed tests include varied ON-time of the motor from 2, 5, 10, 20, 30, and 60 minutes, cyclic tests included repeated drug delivery every 0.5, 1, 3, 12, 24, 36, and 48 hours, and stepped tests included increasing flow rate at increments of 1, 5, and 10 μL every 30 minutes. Multiple MNAs with different dimensions were fabricated, e.g., microneedles with axial lengths of 0.5, 1, and 1.5 mm, and patches with 500-1500 individual microneedles.

### Preclinical Validation of NM Delivery Device

(2R,6R)-HNK confers many benefits as a deliverable drug that make it suitable for evaluating the utility of the wearable MN drug delivery device on measures relevant to PTSD and pain. Primarily, this drug is a ketamine metabolite that retains the beneficial profile of ketamine on assays that are sensitive to novel and currently used therapies for mood disorders and pain conditions. Additionally, (2R,6R)-HNK lacks the negative side effect profile of sedation, ataxia, and abuse liability associated with ketamine.

A forced swim test was performed to confirm that unlike ketamine, (2R,6R)-HNK does not impair gait function significantly. In addition, pilot studies were conducted to assess the antinociceptive potential of (2R,6R)-HNK (10 mg/kg intraperitoneal (i.p.) injection administered for three consecutive days) in response to a noxious thermal stimulus of 50 degrees Celsius on the hot plate apparatus. The latency to lick a hind paw was measured in males before and after (2R,6R)-HNK treatment at 24, 48, and 72 hours post treatment. Additionally, alpha-amino-3-hydroxyl-5-methyl-4-isoxazolepropionic acid (AMPA) receptor antagonist NBQX was found to effectively block the antinociceptive effects of (2R,6R)-HNK. In contrast, pretreatment with opioid antagonist naltrexone failed to block the effects of (2R,6R)-HNK. These data suggest that (2R,6R)-HNK produces its antinociceptive effects through a non-opioid mediated mechanism.

### Example 2

### Introduction

The objective of the present example was to develop and characterize a minimally-invasive, closed-delivery, grooved microneedle array (MNA) for the treatment of chronic pain conditions, as well as post-traumatic stress disorder (PTSD), in a home setting or on the battlefront. World Mental Health Surveys on Trauma and PTSD indicate that only 25-40% of PTSD cases resolve within 1 year. For the majority of subjects, the mean duration of symptoms equated to 6 years across a range of trauma types. Additionally, chronic pain impacts the lives of 20-30% of U.S. adults. The incidence of pain conditions is significantly greater in military service members and veterans, given the likelihood of injury during combat and training. Clinical studies have recently demonstrated that subanesthetic doses of racemic ketamine, administered through infusions, can be used to treat chronic pain conditions and alleviate symptoms of PTSD. Despite the promise ketamine has shown for treating these conditions, its widespread use has been slowed due to the potential for side-effects such as psychotomimetic symptoms, sedation, and dependency. However, recent studies have shown that a ketamine metabolite, 2R,6R-hydroxynorketamine ((2R,6R)-HNK)), can provide similar therapeutic outcomes as ketamine without the potential for negative side-effects typically associated with ketamine infusions. The research for this example aimed to 1) to demonstrate the utility of the MNA device and 2) determine the utility of MNA delivery of (2R,6R)-HNK in practice for PTSD and chronic pain endpoints. By integrating the MNA with sensors to monitor the treatment of the patient, this cutting-edge device provided patient specific therapies for chronic pain and PTSD by allowing the physician to adjust dosage and release rate.

### Materials and Methods

Grooved MNAs were initially assembled with a sodium polyacrylate bead and reservoir as illustrated in FIG. 2A. The proposed design and fabrication process was scalable to realize MNAs with different aspect ratios for both small and large animals and humans. The assembled MNAs were then attached to the skin, where capillary action sampled the underlying fluid through grooved MNs, as illustrated in FIG. 2B. When ISF reached the base of the MNs, the bead expanded in contact with the ISF to sustain the sampling process. Fully expanded beads were then removed from the reservoir for future downstream analysis as illustrated in FIG. 2C.

A single grooved MN was designed using a CAD tool such as SolidWorks®, with the following dimensions: height 1.2 mm, base diameter 0.47 mm, and maximum grooved aperture 0.26 mm. A master array of 4×4 MNs was then fabricated using Nanoscribe (Nanoscribe GmbH), with total width, length, and height MN array dimensions of 3.6 mm, 3.6 mm, and 1.95 mm, respectively. This master MNs patch was replicated by casting PDMS and cured for 3 hours at 60 degrees C., as illustrated in FIG. 7. The resulting PDMS mold was treated with oxygen plasma and then silanized. Bio-compatible/photo-curable hard resin (Dental SG) (Formlabs) was then casted on the PDMS mold and thermally and UV cured in Form Cure (Formlabs) at 60 degrees C. for an hour. The backside reservoir was then fabricated and assembled to hold absorbent bead as further illustrated in FIG. 7.

The sampling kinetics using a skin model made from agarose phantom gel were studied using grooved MN patch. For easy visualization, the skin gel was loaded with Rhodamine B dye, which is sampled into the bead through the grooved MNs, validating the continuous sampling process as illustrated in FIG. 9A. Data of the bead's size increment versus time was collected and analyzed, proving the workability of the proposed grooved MNs as illustrated in FIG. 9B.

In order to demonstrate the passive drug release properties of grooved microneedles, microneedle arrays with different groove dimensions, i.e. a 50 μm, 100 μm, and 200 μm wide channel, as illustrated in FIGS. 13A-13F, respectively, were fabricated. The passive grooved microneedle arrays were designed using Solidworks® and 3D printed using an Asiga Max X27 UV printer and PlasClear V2 resin (Asiga, Alexandria, Australia). To simulate the properties of skin ex vivo, hydrogels comprised of 2% agar (Sigma Aldrich, Burlington, MA) in phosphate buffered saline 1× (Gibco, Waltham, MA) were synthesized. To simulate the drug solution, a 1 mg/ml solution of sulforhodamine B dye (Sigma Aldrich, Burlington, MA) in deionized water was prepared. The microneedle arrays were first primed by siphoning the sulforhodamine B dye solution through the grooves. Then, the passive grooved microneedle arrays were inserted into the agar gels manually and 500 μL of sulforhodamine B dye solution was dispensed into the reservoir for each microneedle array. Each microneedle array was left in its respective agar gel until it had fully eluted the 500 μL of dye solution. Dye solution that did not diffuse into the gel, but rather pooled on the surface of the gel was promptly removed using a Kimwipe (Kimberly-Clark Professional, Dallas, TX).

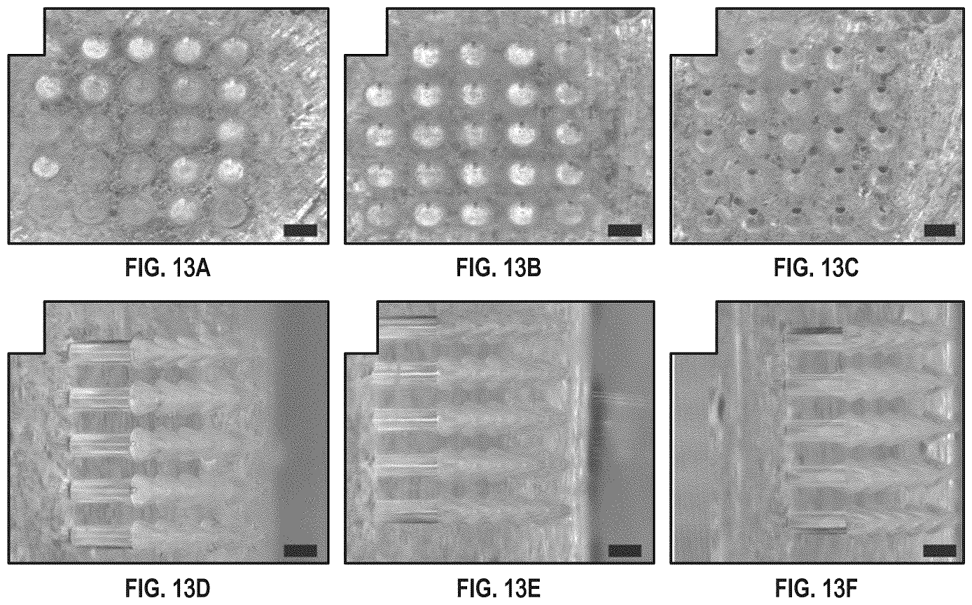

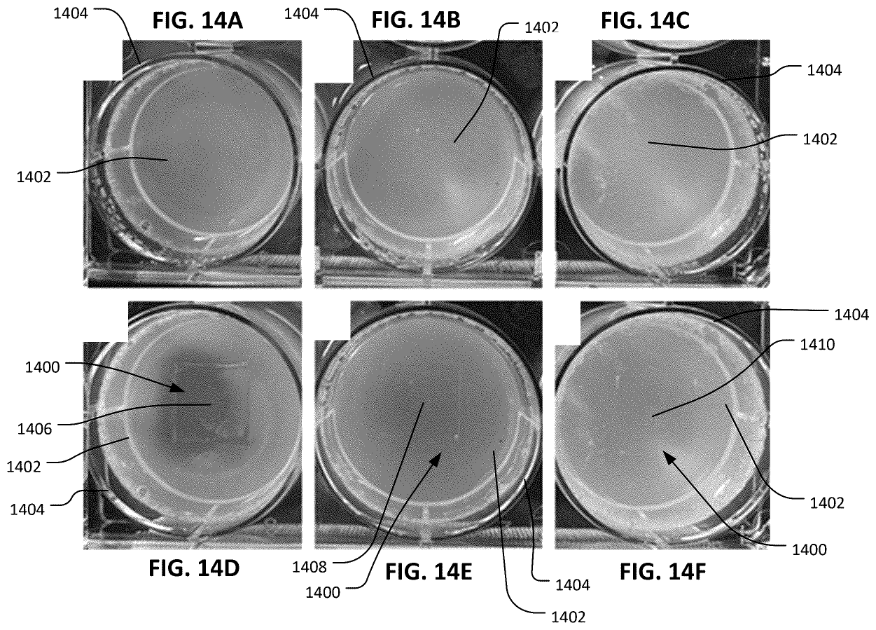

FIGS. 14A-14F illustrate the results of the ex vivo release studies for the passive grooved microneedle arrays. As the microneedle groove diameter was decreased, there was an increase in time to elute the 500 μL dye solution 1400 payload into the gel 1402 contained in the sampling containers 1404. FIG. 14D illustrates a grooved microneedle array 1406 with a channel width of 50 μm in the gel 1402, FIG. 14E illustrates a grooved microneedle array 1408 with a channel width of 100 μm in the gel 1402, and FIG. 14F illustrates a grooved microneedle array 1410 with a channel width of 200 μm in the gel 1402. However, this increase in delivery time led to increased dye flux into the agar gels. This suggests that slower drug elution rates from either the passive or active microneedle arrays may result in improved therapeutic outcomes.

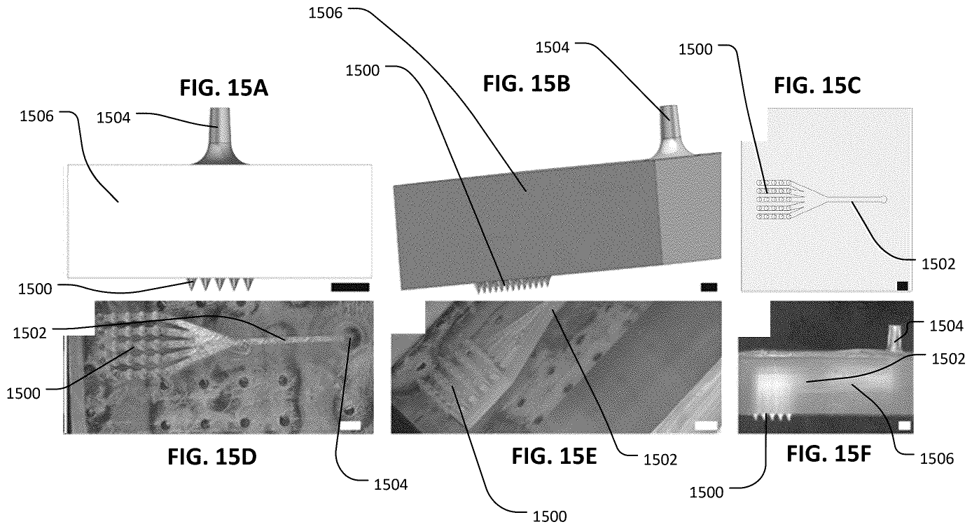

In order to demonstrate the active drug release properties of grooved microneedles, a microneedle array with a 200 μm wide groove was fabricated as illustrated in FIGS. 15A-15F. The active grooved microneedle array 1500 were designed, using Solidworks® as illustrated in the model views FIGS. 15A-15C, to be connected by a 40 μm wide microfluidic channel 1502, which was then run to a fluid inlet 1504 on the opposite side of housing part 1506. The active grooved microneedle array 1500 was then 3D printed using an Asiga Max X27 UV printer and PlasClear V2 resin (Asiga, Alexandria, Australia). To simulate the properties of skin ex vivo, a 10% gelatin eutectogel (made using a deep eutectic solvent system of choline chloride, ethylene glycol, and water mixed in a 1:2:1 molar ratio (Sigma Aldrich, Burlington, MA)) was used. To simulate drug solution, a 1 mg/ml solution of sulforhodamine B dye (Sigma Aldrich, Burlington, MA) in deionized water was prepared. The 3D printed microneedle array was connected via flexible tubing to the outlet of a Takasago piezoelectric micro pump (Model: SDMP302D). The inlet of the Takasago micro pump was then connected via flexible tubing to a 50 ml reservoir of sulforhodamine B dye solution. Then, the active grooved microneedle arrays were inserted into the gelatin eutectogels manually and the voltage source connected to the micropump (Model: HY30005, Mastech) was turned on, initiating fluid flow through the microneedles. The voltage applied to the micropump was 2.5V or 5V, for a slow or fast fluid flow rate, respectively. The release study was run for 30 minutes and any dye solution that did not diffuse into the gel, but rather pooled on the surface of the gel was promptly removed using a Kimwipe (Kimberly-Clark Professional, Dallas, TX).

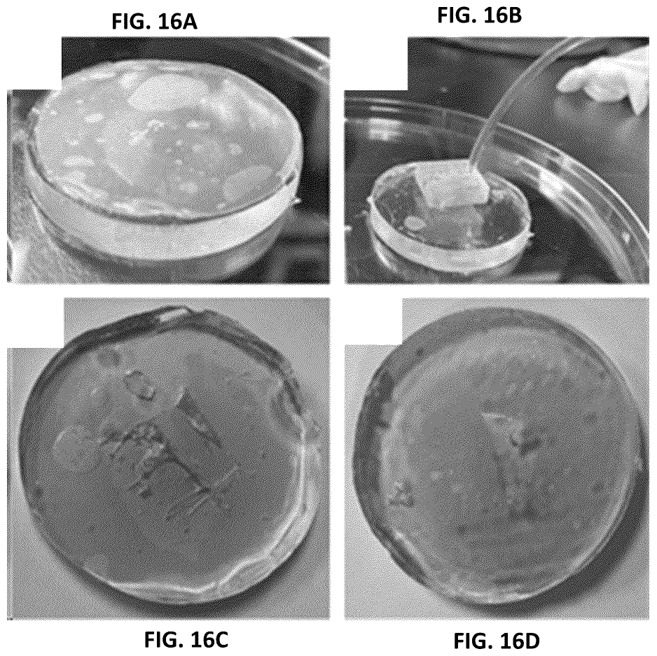

Referring now to FIGS. 16A-16D, the results from the active, electronically controlled grooved microneedle array, with 200 μm wide grooves, are illustrated. By varying the flow rate of the piezoelectric micro pump, different release profiles from the same grooved microneedle patch were achieved, as illustrated in FIG. 16C, in which a 50% flow rate was used, in comparison to FIG. 16D, in which a 100% flow rate was used. Therefore, both the microneedle geometry and the piezoelectric pump speed were adjusted to optimally design the active microneedle arrays for the future delivery of (2R,6R)-HNK.

### Example 3

The use of microneedle arrays is an innovative approach to drug delivery that involves a substrate with submillimeter-sized needles. The proposed system effectively delivers therapeutic compounds into the skin by bypassing the outermost layer, thereby reducing the side effects associated with traditional oral and intravenous drug administration. Hollow-structured microneedles, a type of MNA used for drug release, facilitate the transfer of drugs from the reservoirs to the transdermal layer. However, challenges arise during tissue penetration because the hollow edges can be obstructed by the tissue, leading to drug release failure. Additionally, MNAs that rely on passive drug release lack an active control system, which can result in drug overdose or underdose. The diffusion of drugs into the skin also varies based on the hydrophobic or hydrophilic properties of the drug.

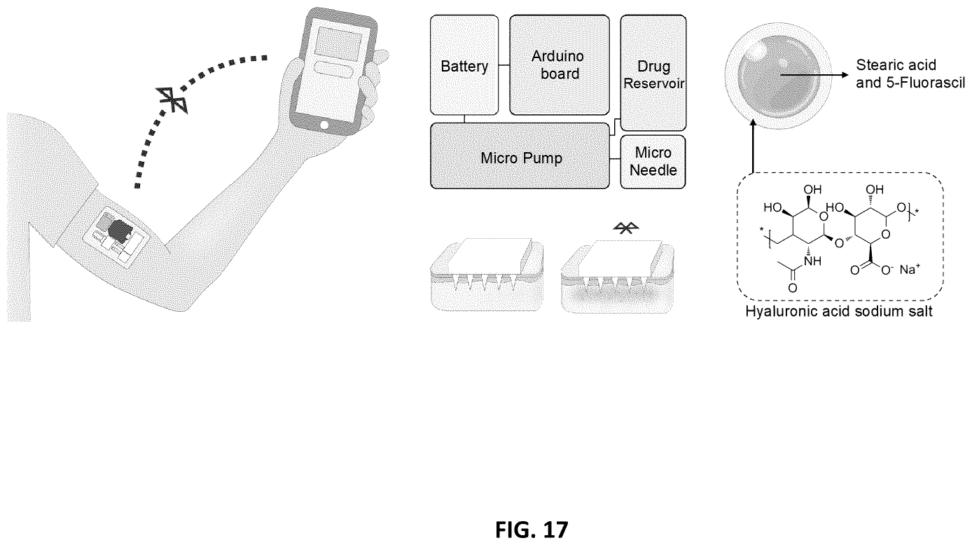

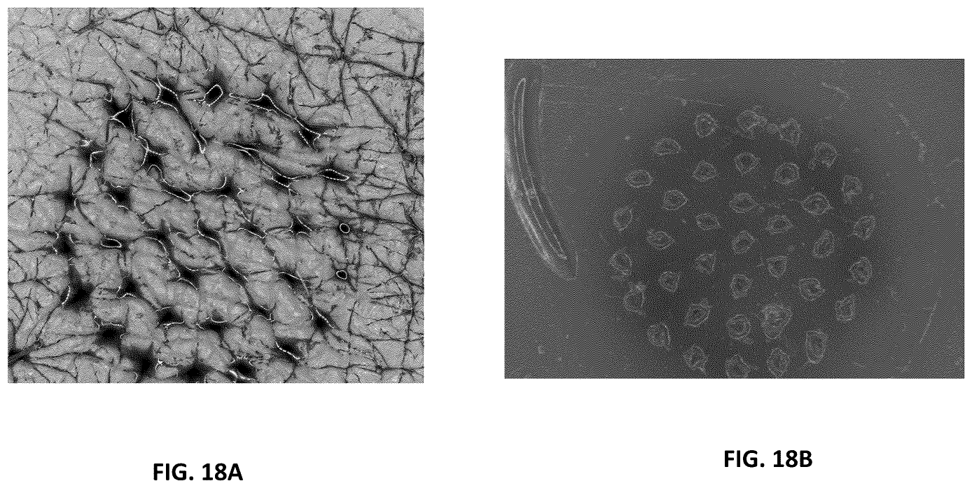

To overcome the limitations of hollow microneedle array (MNA) systems, the present example proposed an integrated, smartphone-controlled, and 3D-printed Hollow-Grooved Microneedle array (HG-MNA) drug delivery system, as illustrated in FIG. 17. This system included a battery powered micro pump, drug reservoir, and microneedle, and features Bluetooth communication for the precise control of drug dosage and system operation. In contrast to previous designs, the HG-MNA fabricated using DLP based 3D printer featured two hollows in each microneedle connected to the groove, which enhanced the drug flow and minimized the risk of hollow blockage during tissue penetration. An example of a microneedle of the proposed HG-MNA is illustrated in FIG. 6A and has a 39.33±2.88 μm tip radius, thus providing sufficient sharpness for effective skin penetration, and a 1.54±0.01 mm height, thus ensuring transdermal reach. The performance of the HG-MNA system was validated using a porcine skin penetration test, which demonstrated successful skin penetration of trypan blue-loaded HG-MNA and drug release confirmed by sulforhodamine B, as illustrated in FIGS. 18A and 18B.

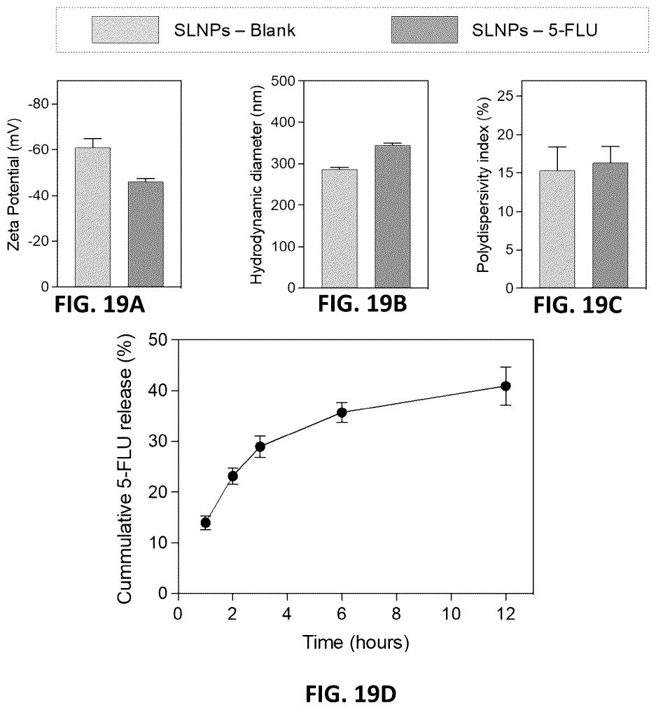

Furthermore, to demonstrate the versatility of the platform, the HG-MNA was loaded with core-shell solid lipid nanoparticles carrying 5-Fluorouracil (5-Fluo) as a model drug for melanoma treatment. The core of the SLNPs comprised stearic acid loaded with 5-Fluo, while the shell comprised hyaluronic acid. The loaded and unloaded nanoparticles exhibited zeta potentials higher than −40 mV, indicating their stability, as illustrated in FIG. 19A. Additionally, they displayed hydrodynamic diameters of 287±4 and 345±5 nm, respectively, with a polydispersity index lower than 20%, confirming their homogeneous size, as illustrated in FIG. 19BC and 19C. Drug release experiments revealed that the SLNs were capable of sustained drug release for 12 hours, as illustrated in FIG. 19D. By combining core-shell nanoparticles and a microneedle platform that allows remote and precise controlled delivery of such nanostructured drug carriers, the present example created new possibilities for advanced personalized medicine.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks, e.g., compact disks and digital video disks, magnetic cassettes, memory cards or sticks, random access memories (RAMs), read only memories (ROMs), and the like.

As used in the claims, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has been described in terms of one or more preferred embodiments, and it should be appreciated that many equivalents, alternatives, variations, and modifications, aside from those expressly stated, are possible and within the scope of the disclosure.

I claim:

1. A drug delivery system, comprising:
a patch including at least one microneedle coupled thereto;
a reservoir coupled to the patch and retaining a fluid containing a drug solution therein; and
a pump coupled to the reservoir,
wherein the at least one grooved microneedle defines a fluid channel therein, and
wherein the pump is configured to direct the fluid containing the drug solution from the reservoir, through the fluid channel of the microneedle, and to a region of interest of a subject.

2. The drug delivery system of claim 1, wherein a first side of the patch includes an adhesive backing.

3. The drug delivery system of claim 1, wherein the at least one microneedle is one of a plurality of microneedles that are arranged in an array on the patch.

4. The drug delivery system of claim 1, wherein the reservoir includes a first reservoir and a second reservoir, and wherein the first reservoir is in fluid communication with the second reservoir.

5. The drug delivery system of claim 4, wherein the pump is configured to direct the fluid containing the drug solution from the second reservoir into the first reservoir.

6. The drug delivery system of claim 4, wherein the second reservoir is disposed within the first reservoir.

7. The drug delivery system of claim 4, wherein a microvalve allows selective fluid communication between the first reservoir from the second reservoir, and
wherein the microvalve is configured to control a flow rate of the fluid containing the drug solution between the first reservoir and the second reservoir.

8. The drug delivery system of claim 7, wherein the microvalve and the pump are in electronic communication with one another.

9. The drug delivery system of claim 7, wherein the microvalve and the micropump are wirelessly connected to a controller via Bluetooth, Wi-Fi, or another radio wireless communication standard.

10. The drug delivery system of claim 9, wherein the controller is configured to selectively and wirelessly actuate the microvalve and the micropump.

11. A system for delivering a fluid containing a drug solution to a region of interest of a subject, comprising:
a patch including at least one microneedle coupled to a first side thereof; and
a reservoir coupled to a second side of the patch,
wherein the at least one microneedle includes a body with a central channel therein, the central channel being in fluid communication with a groove disposed on an exterior side of the body.

12. The system of claim 11, wherein the at least one microneedle defines an axial length between an apex and a base along a longitudinal axis, and wherein the base defines an outer diameter of the microneedle that is less than the axial length.

13. The system of claim 12, wherein the groove defines a groove length along the longitudinal axis, and wherein the groove length is between 30% and 100% of the axial length of the at least one microneedle, expressed as a percentage.

14. The system of claim 11 further comprising a pump coupled to the reservoir and the at least one microneedle, a battery coupled to the micropump, and a controller coupled to the battery.

15. The system of claim 14, wherein the controller is configured to wirelessly operate the pump to direct the fluid containing the drug solution from the reservoir and through the central channel of the microneedle.

16. The system of claim 11, wherein the central channel is one of a plurality of channels disposed within the body of the at least one microneedle.

17. A method of transdermal drug delivery, the method comprising:
placing, on a subject's skin, a microneedle patch including at least one microneedle coupled to a first side of the microneedle patch and a reservoir coupled to a second side of the microneedle patch; and
driving a fluid containing a drug solution from the reservoir, through a fluid channel defined by the at least one microneedle, and to a region of interest in a subject.

18. The method of claim 17, wherein driving the fluid containing the drug solution is performed by one or more of a syringe pump, a vacuum pump, or a micropump.

19. The method of claim 17 further comprising controlling a flow rate of the fluid containing the drug solution with a controller.

20. The method of claim 19, wherein the controller is electronic communication with a microvalve coupled to the reservoir, a micropump coupled to the reservoir, or both.
